(19) United States
(12) Patent Application Publication
(10) Pub. No.: US 2024/0287483 A1
(43) Pub. Date: Aug. 29, 2024

(54) NUCLEIC ACID POLYMERASE FOR INCORPORATING LABELED NUCLEOTIDES

(71) Applicant: Ultima Genomics, Inc., Fremont, CA (US)

(72) Inventors: Daniel MAZUR, San Diego, CA (US); Eileen Collins TOZER, San Diego, CA (US); Theo NIKIFOROV, Carlsbad, CA (US); Denise Sanchez TOPACIO-HALL, Oceanside, CA (US)

(21) Appl. No.: 18/651,565

(22) Filed: Apr. 30, 2024

Related U.S. Application Data
(63) Continuation of application No. PCT/US2022/079074, filed on Nov. 1, 2022.
(60) Provisional application No. 63/263,440, filed on Nov. 2, 2021.

(51) Int. Cl. C12N 9/12; C12Q 1/6825

(52) U.S. Cl. CPC C12N 9/1252; C12Q 1/6825; C12Y 207/07007

(57) ABSTRACT

Mutant nucleic acid polymerases are described herein. Also described are methods for screening or assaying the mutant nucleic acid polymerases.

### CROSS-REFERENCE TO RELATED APPLICATION

This application is a Continuation application of International Application No. PCT/US2022/079074, filed internationally on Nov. 1, 2022, which claims the priority benefit of U.S. Provisional Patent Application Ser. No. 63/263,440, filed on Nov. 2, 2021, the contents of each are incorporated herein by reference in their entirety.

### REFERENCE TO AN ELECTRONIC SEQUENCE LISTING

The contents of the electronic sequence listing (165272001801SEQLIST.xml; Size: 38,062 bytes; and Date of Creation: Apr. 25, 2024) is herein incorporated by reference in its entirety.

### FIELD OF THE INVENTION

Described herein are nucleic acid polymerases. The polymerases can incorporate a labeled nucleotide into an extending nucleic acid molecule. Also described are methods of using the nucleic acid polymerase, for example in a nucleic acid sequencing method, and methods of making the nucleic acid polymerase.

### BACKGROUND

Polymerases can catalyze the polymerization of biomolecules (e.g., nucleotides or amino acids) into biopolymers (e.g., nucleic acids or peptides). For example, polymerases that can polymerize nucleotides into nucleic acids, particularly in a template-dependent fashion, are useful in recombinant DNA technology and nucleic acid sequencing applications. Many nucleic acid sequencing methods monitor nucleotide incorporations during in vitro template-dependent nucleic acid synthesis catalyzed by a polymerase. Nucleic acid libraries created using such polymerases can be used in a variety of downstream processes, such as genotyping, nucleotide polymorphism (SNP) analysis, copy number variation analysis, epigenetic analysis, gene expression analysis, hybridization arrays, analysis of gene mutations including but not limited to detection, prognosis and/or diagnosis of disease states, detection and analysis of rare or low frequency allele mutations, and nucleic acid sequencing including but not limited to de novo sequencing or targeted resequencing.

When performing polymerase-dependent nucleic acid synthesis or amplification, it can be useful to modify the polymerase (e.g., via mutation or chemical modifications) so as to alter (e.g., enhance) its catalytic properties. Polymerase performance in various biological assays involving nucleic acid synthesis can be limited by the kinetic behavior of the polymerase towards nucleotide substrates. For example, analysis of polymerase activity can be complicated by undesirable behavior such as the tendency of a given polymerase to dissociate from the template, to bind and/or incorporate the incorrect (e.g., non-Watson-Crick base-paired, nucleotide), or to release the correct (e.g., Watson-Crick based paired, nucleotide) without incorporation. Desirable catalytic properties can be enhanced via suitable selection, engineering and/or modification of a polymerase of choice. For example, such modification can be performed to favorably alter the polymerase's rate of nucleotide incorporation, rate of nucleotide misincorporation, affinity of binding to template, processivity or average read length. Such alterations can increase the amount of sequence information obtained from a single sequencing reaction. There remains a need in the art for improved polymerase compositions exhibiting altered (e.g., increased) processivity, read length (e.g., including error-free read length), and/or affinity for DNA template. Such polymerase compositions can be useful in a wide variety of assays involving polymerase-dependent nucleic acid synthesis, including nucleic acid sequencing and production of nucleic acid libraries.

### BRIEF SUMMARY OF THE INVENTION

Described herein are mutant polymerase compositions and methods of making same. Also disclosed herein are methods and systems for screening mutant polymerases that recognize certain nucleotide substrates (e.g., nucleotide-linker-dye conjugates that do not include terminator moieties). Further disclosed are methods and systems for evaluating activities of mutant polymerases.

Described herein is a nucleic acid polymerase, comprising an amino acid sequence having at least 95% sequence identity to SEQ ID NO:1, wherein the polymerase comprises a mutation, relative to SEQ ID NO: 1, at an amino acid position selected from the group consisting of 495, 515, 526-534, 538, 549, 551-568, 570-590, 609-630, 635-637, 640, 655-663, 674, 681, 683, 688, 689, 701, 703-720, 728-730, 743, 761, 770-829, 872, and 873, wherein the amino acid position numbering is according to SEQ ID NO: 2. In some instances, the mutation is at amino acid position 495, 515, 529, 531, 538, 549, 558, 559, 561, 562, 563, 564, 565, 566, 567, 568, 572, 573, 575, 579, 580, 583, 622, 623, 628, 629, 630, 635, 636, 637, 640, 655, 660, 661, 662, 674, 681, 683, 688, 689, 701, 703, 704, 707, 710, 713, 716, 718, 743, 761, 780, 782, 785, 786, 788, 800, 806, 815, 819, 826, 827, 872, or 873. In some implementations, the mutation is V495F, E515K, N529R, P531Q, L538F, K549I, A558M, D559K, D559R, D559S, L561I, E562N, K563G, L564V, A565R, P566L, Y567H, H568P, E572D, N573E, L575I, Q579N, L580D, L580G, L583N, L583V, N622G, E623Y, I628L, I628M, R629A, L630D, L630P, K635S, I636M, I636L, R637M, F640V, S655N, R660C, V661I, L662M, A674V, I681V, T683K, T683V, D688M, I689F, I689Q, I689V, I689Y, M701L, R703A, R703H, R703P, Q704I, A707Y, F710Y, V713M, I716Q, D718A, D718K, F743R, F743Y, G761E, N780K, N782R, N782V, S785R, F786R, E788R, A800G, K806E, K806M, L815F, R819K, R819M, R819P, R819V, L826Y, Q827D, W872H, or Y873D. In some implementations, the polymerase comprises at least two mutations, relative to SEQ ID NO: 1, at amino acid positions selected from the group consisting of 495, 515, 526-534, 538, 549, 551-568, 570-590, 609-630, 635-637, 640, 655-663, 674, 681, 683, 688, 689, 701, 703-720, 728-730, 743, 761, 770-829, 872, and 873. In some implementations, the at least two mutations are at amino acid positions 495, 515, 529, 531, 538, 549, 558, 559, 561, 562, 563, 564, 565, 566, 567, 568, 572, 573, 575, 579, 580, 583, 622, 623, 628, 629, 630, 635, 636, 637, 640, 655, 660, 661, 662, 674, 681, 683, 688, 689, 701, 703, 704, 707, 710, 713, 716, 718, 743, 761, 780, 782, 785, 786, 788, 800, 806, 815, 819, 826, 827, 872, or 873. In some implementations, the at least two mutations comprise two or more of V495F, E515K, N529R, P531Q, L538F, K549I, A558M, D559K, D559R, D559S, L561I, E562N, K563G, L564V, A565R, P566L, Y567H, H568P, E572D, N573E, L575I, Q579N, L580D, L580G, L583N, L583V, N622G, E623Y, I628L, I628M, R629A, L630D, L630P, K635S, I636M, I636L, R637M, F640V, S655N, R660C, V661I, L662M, A674V, I681V, T683K, T683V, D688M, I689F, I689Q, I689V, I689Y, M701L, R703A, R703H, R703P, Q704I, A707Y, F710Y, V713M, I716Q, D718A, D718K, F743R, F743Y, G761E, N780K, N782R, N782V, S785R, F786R, E788R, A800G, K806E, K806M, L815F, R819K, R819M, R819P, R819V, L826Y, Q827D, W872H, or Y873D. In some implementations, the amino acid sequence comprises at least 95% identity to SEQ ID NO: 2. In some implementations, the amino acid sequence further comprises a DNA binding domain. In some implementations, the DNA binding domain comprises a sequence at least 95% identical to SEQ ID NO: 4 or SEQ ID NO: 5. In some implementations, the DNA binding domain comprises a sequence of SEQ ID NO: 4 or SEQ ID NO: 5. In some implementations, the DNA binding domain is attached to the N-terminus of a polymerase domain. In some implementations, the DNA binding domain is attached to the N-terminus of the polymerase domain through an amino acid linker.

Also described herein is a nucleic acid polymerase, comprising an amino acid sequence having at least 95% sequence identity to SEQ ID NO: 3, wherein the polymerase comprises a mutation, relative to SEQ ID NO: 3, at an amino acid position selected from the group consisting of 495, 515, 526-534, 538, 549, 551-568, 570-590, 609-630, 635-637, 640, 655-663, 674, 681, 683, 688, 689, 701, 703-720, 728-730, 743, 761, 770-829, 872, and 873, wherein the amino acid position numbering is according to SEQ ID NO: 2. In some implementations, the mutation is at amino acid position 495, 515, 529, 531, 538, 549, 558, 559, 561, 562, 563, 564, 565, 566, 567, 568, 572, 573, 575, 579, 580, 583, 622, 623, 628, 629, 630, 635, 636, 637, 640, 655, 660, 661, 662, 674, 681, 683, 688, 689, 701, 703, 704, 707, 710, 713, 716, 718, 743, 761, 780, 782, 785, 786, 788, 800, 806, 815, 819, 826, 827, 872, or 873, wherein the amino acid position numbering is according to SEQ ID NO: 2. In some implementations, the mutation is V495F, E515K, N529R, P531Q, L538F, K549I, A558M, D559K, D559R, D559S, L561I, E562N, K563G, L564V, A565R, P566L, Y567H, H568P, E572D, N573E, L575I, Q579N, L580D, L580G, L583N, L583V, N622G, E623Y, I628L, I628M, R629A, L630D, L630P, K635S, I636M, I636L, R637M, F640V, S655N, R660C, V661I, L662M, A674V, I681V, T683K, T683V, D688M, I689F, I689Q, I689V, I689Y, M701L, R703A, R703H, R703P, Q704I, A707Y, F710Y, V713M, I716Q, D718A, D718K, F743R, F743Y, G761E, N780K, N782R, N782V, S785R, F786R, E788R, A800G, K806E, K806M, L815F, R819K, R819M, R819P, R819V, L826Y, Q827D, W872H, or Y873D, wherein the amino acid position numbering is according to SEQ ID NO: 2. In some implementations, the polymerase comprises at least two mutations, relative to SEQ ID NO: 1, at amino acid positions selected from the group consisting of 495, 515, 526-534, 538, 549, 551-568, 570-590, 609-630, 635-637, 640, 655-663, 674, 681, 683, 688, 689, 701, 703-720, 728-730, 743, 761, 770-829, 872, and 873, wherein the amino acid position numbering is according to SEQ ID NO: 2. In some implementations, the at least two mutations are at amino acid positions 495, 515, 529, 531, 538, 549, 558, 559, 561, 562, 563, 564, 565, 566, 567, 568, 572, 573, 575, 579, 580, 583, 622, 623, 628, 629, 630, 635, 636, 637, 640, 655, 660, 661, 662, 674, 681, 683, 688, 689, 701, 703, 704, 707, 710, 713, 716, 718, 743, 761, 780, 782, 785, 786, 788, 800, 806, 815, 819, 826, 827, 872, or 873, wherein the amino acid position numbering is according to SEQ ID NO: 2. In some implementations, the at least two mutations comprise two or more of V495F, E515K, N529R, P531Q, L538F, K549I, A558M, D559K, D559R, D559S, L561I, E562N, K563G, L564V, A565R, P566L, Y567H, H568P, E572D, N573E, L575I, Q579N, L580D, L580G, L583N, L583V, N622G, E623Y, I628L, I628M, R629A, L630D, L630P, K635S, I636M, I636L, R637M, F640V, S655N, R660C, V661I, L662M, A674V, I681V, T683K, T683V, D688M, I689F, I689Q, I689V, I689Y, M701L, R703A, R703H, R703P, Q704I, A707Y, F710Y, V713M, I716Q, D718A, D718K, F743R, F743Y, G761E, N780K, N782R, N782V, S785R, F786R, E788R, A800G, K806E, K806M, L815F, R819K, R819M, R819P, R819V, L826Y, Q827D, W872H, or Y873D, wherein the amino acid position numbering is according to SEQ ID NO: 2.

In some implementations of the above, the polymerase comprises a mutation at amino acid position 628, wherein the amino acid position numbering is according to SEQ ID NO: 2. In some implementations, the polymerase comprises a I628L mutation, wherein the amino acid position numbering is according to SEQ ID NO: 2.

In some implementations of the above, the polymerase comprises a 785R mutation, wherein the amino acid position numbering is according to SEQ ID NO: 2.

In some implementations of the above, the polymerase comprises a mutation at amino acid position 630, wherein the amino acid position numbering is according to SEQ ID NO: 2. In some implementations, the polymerase comprises a L630D mutation, wherein the amino acid position numbering is according to SEQ ID NO: 2. In some implementations, the polymerase comprises a L630P mutation, wherein the amino acid position numbering is according to SEQ ID NO: 2.

In some implementations of the above, the polymerase comprises a mutation at amino acid position 785, wherein the amino acid position numbering is according to SEQ ID NO: 2. In some implementations, the polymerase comprises an S785R mutation, wherein the amino acid position numbering is according to SEQ ID NO: 2.

In some implementations of the above, the polymerase comprises a mutation at amino acid position 827, wherein the amino acid position numbering is according to SEQ ID NO: 2. In some implementations, the polymerase comprises a Q827D mutation, wherein the amino acid position numbering is according to SEQ ID NO: 2.

Also described herein is a composition comprising a polymerase according to any of the above and an aqueous solution. In some implementations, the method further comprises nucleotides and a nucleic acid hybrid comprising a target nucleic acid molecule hybridized to a sequencing primer. In some implementations, at least a portion of the nucleotides are labeled nucleotides. In some implementations, the labeled nucleotides comprise a fluorescent label.

Further described herein is a nucleic acid molecule encoding the any one of the above polymerases. Also described is an expression vector comprising said nucleic acid molecule. Further described is host cell, comprising said expression vector. Also described is method of making a polymerase, comprising culturing said host cell; expressing, using the host cell, the polymerase; and isolating the polymerase.

Also described is a method, comprising providing in a reaction mixture (i) a nucleic acid molecule, (ii) a labeled nucleotide, and (iii) the mutant polymerase according to any of the above, under conditions sufficient to extend the nucleic acid molecule with the labeled nucleotide. In some implementations, said labeled nucleotide comprises a fluorescent dye.

Further described is a method, comprising contacting a labeled nucleotide with the mutant polymerase of any of the above and extending a nucleic acid molecule to incorporate the labeled nucleotide. In some implementations, said labeled nucleotide comprises a fluorescent dye.

In one aspect, the disclosure relates to one or more modified polymerases, where each of the one or more modified polymerases contain at least one amino acid mutation in comparison with a reference polymerase. In some embodiments, the disclosure relates to one or more modified DNA or RNA polymerases. In some embodiments, the disclosure relates to a modified polymerase for use in nucleic acid sequencing, including next-generation sequencing. In some embodiments, the disclosure relates to a modified polymerase for use in generated nucleic acid libraries and/or nucleic acid templates. In some embodiments, the disclosure relates to kits comprising one or more of the modified polymerases. In some embodiments, the modified polymerase is a mutant BST polymerase bearing one or more single nucleotide mutations. In some embodiments, the modified polymerase comprises a truncated polymerase domain without an exonuclease domain. In some embodiments, the modified polymerase is a hybrid protein comprising a truncated polymerase domain and a DNA binding domain (e.g., Mcu7, SSO7d domain, etc.). In some embodiments, the modified polymerase further comprises a linker domain between the polymerase and DNA binding domain.

In some embodiments, methods are described herein that use FRET-based homogeneous polymerase assays for monitoring nucleotide incorporation reactions that utilize specific combinations of template oligonucleotide sequence design and fluorescence probes to qualitatively and/or quantitatively evaluate polymerase nucleotide incorporation rates and polymerase processivities. In some embodiments, the disclosed methods may be used to monitor nucleotide incorporation reactions and evaluate polymerase nucleotide incorporation rates, e.g., for specific labeled, nucleotides. In some embodiments, the methods may be used to monitor nucleotide incorporation reactions and evaluate polymerase processivities for processing and replicating specific template sequences, e.g., homopolymer sequences to determine a maximum homopolymer sequence length for which a given polymerase is capable of efficiently synthesizing a complementary oligonucleotide strand. In some embodiments, the disclosed methods may be used to monitor nucleotide incorporation reactions and evaluate nucleotide incorporation rates e.g., using specific, labeled nucleotides. In some embodiments, the methods may be used to screen libraries of mutant polymerases to rank order and select mutant polymerases on the basis of their nucleotide incorporation rates (e.g., for specific labeled nucleotides), and/or processivities (e.g., for specific template sequences such as homopolymer sequences).

In some embodiments, methods are described herein that use FRET-based homogeneous polymerase assays for monitoring nucleotide incorporation reactions that utilize specific combinations of template oligonucleotide sequence design and fluorescence probes to qualitatively and/or quantitatively evaluate polymerase nucleotide misincorporation rates. In some embodiments, the disclosed methods may be used to monitor nucleotide misincorporation reactions and evaluate polymerase nucleotide misincorporation rates, e.g., for specific labeled, nucleotides. In some embodiments, the methods may be used to screen libraries of mutant polymerases to rank order and select mutant polymerases on the basis of their nucleotide misincorporation rates (e.g., for specific labeled nucleotides).

### INCORPORATION BY REFERENCE

All publications, patents, and patent applications mentioned in this specification are herein incorporated by reference in their entirety to the same extent as if each individual publication, patent, or patent application was specifically and individually indicated to be incorporated by reference in its entirety. In the event of a conflict between a term herein and a term in an incorporated reference, the term herein controls.

### BRIEF DESCRIPTION OF THE DRAWINGS

Various aspects of the disclosed methods are set forth with particularity in the appended claims. A better understanding of the features and advantages of the methods will be obtained by reference to the following detailed description of illustrative embodiments and the accompanying drawings, of which:

FIG. 1 provides a non-limiting schematic illustration of a FRET-based polymerase assay as described herein.

FIG. 2 provides a non-limiting schematic illustration of a FRET-based polymerase assay as described herein.

FIG. 3 provides a non-limiting example of fluorescence intensity plots for a polymerase assay as described herein.

FIG. 4 provides a non-limiting example of fluorescence intensity plots for a polymerase assay as described herein.

FIG. 5 provides a non-limiting example of fluorescence intensity plots for a polymerase assay as described herein.

FIG. 6 provides a non-limiting example of fluorescence intensity plots for a polymerase assay as described herein.

FIG. 7 provides a non-limiting example of fluorescence intensity plots for a polymerase assay as described herein.

FIG. 8 provides a non-limiting example of fluorescence intensity plots for a polymerase assay as described herein.

FIG. 9 provides a non-limiting example of fluorescence intensity plots for a polymerase assay as described herein.

FIG. 10 provides a non-limiting example of fluorescence intensity plots for a polymerase assay as described herein.

FIG. 11 provides a non-limiting example of fluorescence intensity plots for a polymerase assay as described herein.

FIG. 12 provides a non-limiting example of fluorescence intensity plots for a polymerase assay as described herein.

FIGS. 13A and 13B illustrate examples of multiple-label incorporation and misincorporation FRET-based assays

FIGS. 14A and 14B illustrate examples of multiple-label incorporation and misincorporation FRET-based assays.

FIGS. 15A and 15B illustrate examples of multiple-label incorporation and misincorporation FRET-based assays.

FIGS. 16A and 16B illustrate examples of multiple-label incorporation and misincorporation FRET-based assays.

FIGS. 17A and 17B illustrate examples of multiple-label incorporation and misincorporation FRET-based assays.

FIG. 18 illustrates a non-limiting example of a method for making and assaying a mutant polymerase construct.

FIGS. 19A and 19B illustrates non-limiting examples of mutant polymerase constructs.

FIG. 20 illustrates the sequence of a non-limiting example of BST DNA polymerase domain from Bacillus stearothermophilus, showing locations of some of the amino acid residues subject to mutagenesis analysis.

FIG. 21 illustrates exemplary sequencing results for a mutant polymerase.

FIG. 22 illustrates exemplary sequencing results for a mutant polymerase.

FIG. 23 provides a non-limiting example of fluorescence intensity plots for a polymerase assay as described herein.

FIG. 24 provides a non-limiting example of fluorescence intensity plots for a polymerase assay as described herein.

### DETAILED DESCRIPTION

In some instances, the disclosure relates to polymerase compositions and methods of making and using same.

In some instances, the methods described herein comprise: forming a reaction mixture comprising: (i) a template oligonucleotide sequence comprising a primer binding site, one or more nucleotide residues (e.g., a sequence segment comprising a single nucleotide residue, or two or more nucleotide residues) comprising the same base, and a first member of a fluorescence probe pair; (ii) a primer sequence configured to hybridize to the primer binding site; (iii) a first nucleotide set where a member of the first nucleotide set comprises a second member of the fluorescence probe pair, and where the first nucleotide set comprises one or more nucleotides that are not complementary to the one or more nucleotide residues; (iv) a second nucleotide set comprising one or more nucleotides that are complementary to the one or more nucleotide residues; and (v) a polymerase; and detecting a presence or absence of a fluorescence resonance energy transfer (FRET) signal, where a change in the FRET signal indicates that the polymerase has incorporated nucleotides of the first nucleotide set or has incorporated nucleotides of the second nucleotide set to extend the primer sequence through the one or more nucleotide residues.

In some instances, for example, the methods comprise: forming a reaction mixture comprising: (i) a template oligonucleotide sequence comprising a primer binding site, one or more nucleotide residues (e.g., a sequence segment comprising a single nucleotide residue, or two or more nucleotide residues comprising the same base or a mixture of different bases), and a nucleotide residue at a 5′ end of the one or more nucleotide residues that is different from the one or more nucleotides (e.g., a nucleotide residue comprising a different base from those present in the sequence segment comprising the one or more nucleotide residues); (ii) a primer sequence configured to hybridize to the primer binding site; (iii) a first nucleotide set wherein each member of the set comprises a first member of a fluorescence probe pair, and wherein the first nucleotide set comprises one or more nucleotides that are complementary to the one or more nucleotide residues; (iv) a second nucleotide comprising a second member of the fluorescence probe pair, wherein the second nucleotide comprises a nucleotide that is complementary to the nucleotide residue at the 5′ end of the one or more nucleotide residues; and (v) a polymerase; and detecting a presence or absence of a fluorescence resonance energy transfer (FRET) signal, wherein a change in the FRET signal indicates that the polymerase has incorporated nucleotides of the first nucleotide set and the second nucleotide to extend the primer sequence through the one or more nucleotide residues and the nucleotide residue at the 5′end of the one or more nucleotide residues.

In some instances, as another example, the methods comprise: forming a reaction mixture comprising: (i) a template oligonucleotide sequence comprising a primer binding site, one or more nucleotide residues, a nucleotide residue at a 5′ end of the one or more nucleotide residues that is different from the one or more nucleotides, and a first fluorescence probe; (ii) a primer sequence configured to hybridize to the primer binding site; (iii) a first nucleotide set wherein each member of the set comprises a second fluorescence probe, and wherein the first nucleotide set comprises one or more nucleotides that are complementary to the one or more nucleotide residues; (iv) a second nucleotide comprising a third fluorescence probe, wherein the second nucleotide comprises a nucleotide that is complementary to the nucleotide residue at the 5′ end of the one or more nucleotide residues; and (v) a polymerase; and detecting a presence or absence of a fluorescence resonance energy transfer (FRET) signal, wherein a change in the FRET signal indicates that the polymerase has incorporated nucleotides the first nucleotide set and the second nucleotide to extend the primer sequence through the one or more nucleotide residues and the nucleotide residue at the 5′end of the one or more nucleotide residues.

In some instances, a sequence segment comprising the one or more nucleotide residues may be varied and may range in length from 1 nucleotide residue to about 10 nucleotide residues, or longer.

In some instances, the one or more nucleotide residues may comprise, for example, a single nucleotide residue, a homopolymer sequence, a short tandem repeat sequence comprising a dinucleotide, trinucleotide, or longer repeat pattern. In some instances, for example, the one or more nucleotide residues may comprise a short tandem repeat sequence that has a repeat pattern of from 2 to 16 nucleotides in length and include from 2 to 10 repeats of the repeat pattern. In some instances, the one or more nucleotide residues may comprise a defined sequence, an arbitrary or random sequence, a partially-random sequence (e.g., a mix of defined and random subsequences), or non-random sequence of nucleotide residues ranging from 2 to 50 nucleotide residues in length.

In some instances, the methods comprise the use of a fluorescence probe pair, e.g., a donor-acceptor pair or a donor—quencher pair. In other instances, the methods comprise the use of a series of fluorescence probes, e.g., a first, second, and third fluorophore. In some instances, the first and second fluorophores comprise a fluorescence donor—acceptor pair or a fluorescence donor-quencher pair. In some instances, the second and third fluorophores comprise a fluorescence donor-acceptor pair or a fluorescence donor—quencher pair. In some instances, the first and third fluorophores comprise a fluorescence donor—acceptor pair or a fluorescence donor—quencher pair. Depending on the combination of fluorescence donors, acceptors, and/or quencher used, the successful incorporation of labeled nucleotides by the polymerase to extend the primer sequence may result in either an increase or a decrease of a fluorescence resonance energy (FRET) signal compared to a baseline signal (e.g., a background signal or a signal measured prior to addition of a reaction mixture component such as the polymerase). In some instances, one may additional excite one or more of the fluorescence probes with light of a suitable wavelength and measure a fluorescence emission intensity arising therefrom.

In one aspect, the methods may be used to monitor nucleotide incorporation reactions and evaluate polymerase nucleotide incorporation rates (e.g., for labeled nucleotides) in order to select an optimal polymerase and/or an optimal combination of polymerase and type of labeled nucleotide for a given application (e.g., nucleic acid amplification or sequencing).

In another aspect, the methods may be used to monitor nucleotide incorporation reactions and evaluate polymerase processivities for processing and replicating specific types of sequences (e.g., homopolymer sequences) to determine, for example, if there is a maximum sequence length (e.g., a maximum homopolymer sequence length) for which a given polymerase is capable of efficiently synthesizing a complementary oligonucleotide strand, or to determine an ability of the polymerase to sustain continuous primer extension reactions using labeled nucleotides.

In yet another aspect, the methods may be used to screen libraries of mutant polymerases to rank order and select mutant polymerases on the basis of their nucleotide incorporation rates (e.g., for labeled nucleotides) and/or processivities (e.g., for homopolymer sequences). In some instances, for example, the methods may be used to screen a library of mutant polymerases and select a mutant polymerase that is more efficient than a corresponding wild-type polymerase at incorporating labeled and/or non-labeled nucleotides into a growing nucleic acid strand. In some instances, the methods may be used to screen a library of mutant polymerases and select a mutant polymerase that is less likely than a corresponding wild-type polymerase to mis-incorporate labeled and/or non-labeled nucleotides during nucleic acid sequencing.

The disclosed FRET-based assay methods used to identify polymerase mutants described herein are uniquely suited to the screening of various polymerases and/or combinatorial libraries of polymerase mutants for their sequential incorporation ability of or for their misincorporation activity with dye-labeled nucleotides and process homopolymer sequences. Both of these properties are required for the successful application of polymerases to nucleic acid amplification and sequencing applications.

### Definitions

Unless otherwise defined, all of the technical terms used herein have the same meaning as commonly understood by one of ordinary skill in the art in the field to which this disclosure belongs.

As used in this specification and the appended claims, the singular forms “a”, “an”, and “the” include plural references unless the context clearly dictates otherwise. Any reference to “or” herein is intended to encompass “and/or” unless otherwise stated.

As used herein, the terms “comprising” (and any form or variant of comprising, such as “comprise” and “comprises”), “having” (and any form or variant of having, such as “have” and “has”), “including” (and any form or variant of including, such as “includes” and “include”), or “containing” (and any form or variant of containing, such as “contains” and “contain”), are inclusive or open-ended and do not exclude additional, un-recited additives, components, integers, elements, or method steps.

As used herein, the term “about” a number refers to that number plus or minus 10% of that number. The term “about” when used in the context of a range refers to that range minus 10% of its lowest value and plus 10% of its greatest value.

As used herein, the term “nucleotide” generally refers to a substance including a base (e.g., a nucleobase), sugar moiety, and phosphate moiety. A nucleotide may comprise a free base with attached phosphate groups. A substance including a base with three attached phosphate groups may be referred to as a nucleoside triphosphate. When a nucleotide is being added to a growing nucleic acid molecule strand, the formation of a phosphodiester bond between the proximal phosphate of the nucleotide to the growing chain may be accompanied by hydrolysis of a high-energy phosphate bond with release of the two distal phosphates as a pyrophosphate. The nucleotide may be naturally occurring or non-naturally occurring (e.g., a nucleotide analog that is a modified, synthesized, or engineered nucleotide). A naturally occurring nucleotide may include a canonical base (e.g., A, C, G, T, or U). A nucleotide analog may not be naturally occurring or may include a non-canonical base (e.g., an alternative base). The nucleotide analog may include a modified polyphosphate chain (e.g., triphosphate coupled to a fluorophore). The nucleotide analog may comprise a label. The nucleotide analog may be terminated (e.g., reversibly terminated). Nucleotide analogs that may be used in accordance with embodiments of this disclosure are described, for example, in U.S. patent application Ser. No. 17/150,659, which is hereby incorporated by reference in its entirety.

The terms “label,” “tag,” or “dye” are used interchangeably herein, and generally refer to a moiety that is capable of coupling with a species, such as, for example a nucleotide analog. A label may include an affinity moiety. In some cases, a label may be a detectable label that emits a signal (or reduces an already emitted signal) that can be detected (e.g., a fluorescent tag). In some cases, such a signal may be indicative of incorporation of one or more nucleotides or nucleotide analogs. In some cases, a label may be coupled to a nucleotide or nucleotide analog, which nucleotide or nucleotide analog may be used in a primer extension reaction. In some cases, the label may be coupled to a nucleotide analog after a primer extension reaction. The label, in some cases, may be reactive specifically with a nucleotide or nucleotide analog. Coupling may be covalent or non-covalent (e.g., via ionic interactions, Van der Waals forces, etc.). In some cases, coupling may be via a linker, which may be cleavable, such as photo-cleavable (e.g., cleavable under ultra-violet light), chemically-cleavable (e.g., via a reducing agent, such as dithiothreitol (DTT), tris(2-carboxyethyl)phosphine (TCEP), or tris(hydroxypropyl)phosphine (THP)), or enzymatically cleavable (e.g., via an esterase, lipase, peptidase, or protease). As disclosed herein, the terms cleavable and excisable are used interchangeably. In some cases, the label may be luminescent, that is, fluorescent or phosphorescent. Labels may be quencher molecules. Dyes, quenchers, and labels may be incorporated into nucleic acid sequences.

As used herein a “fluorescence donor” is a fluorophore, quantum dot, or other fluorescent tag that is capable of transferring excited state energy via a radiationless transfer mechanism to a suitable acceptor molecule, e.g., a fluorescence acceptor or fluorescence quencher molecule.

As used herein a “fluorescence acceptor” is a fluorophore, quantum dot, or other fluorescent tag, that is capable of accepting transferred excited state energy and re-emitting all or a portion of it as fluorescence.

As used herein a “fluorescence quencher” is a fluorophore, quantum dot, or other tag molecule that is capable of accepting transferred excited state energy and dissipating all or a portion of the excess energy without re-emitting it as fluorescence. Quenchers, in general, are molecules that can reduce an emitted signal. For example, a template nucleic acid molecule may be designed to emit a detectable signal. Incorporation of a nucleotide or nucleotide analog comprising a quencher (e.g., a fluorescence quencher) can reduce or eliminate the signal (e.g., a fluorescent signal), which reduction or elimination is then detected.

As used herein, the term “excitation wavelength” refers to the wavelength of light used to excite a fluorescent label (e.g., a fluorophore, a fluorescence donor, a fluorescence acceptor, a quantum dot, or a dye molecule) and generate fluorescence. Although the excitation wavelength is typically specified as a single wavelength, e.g., 620 nm, it will be understood by those of skill in the art that this specification refers to a wavelength range or excitation filter band-pass that is centered on the specified wavelength. For example, in some instances, light of the specified excitation wavelength comprises light of the specified wavelength ±2 nm, ±5 nm, ±10 nm, ±20 nm, ±40 nm, ±80 nm, or more. In some instances, the excitation wavelength used may or may not coincide with the absorption peak maximum of the fluorescent indicator.

As used herein, the term “emission wavelength” refers to the wavelength of light emitted by a fluorescent label (e.g., a fluorophore, a fluorescence donor, a fluorescence acceptor, a quantum dot, or a dye molecule) upon excitation by light of an appropriate wavelength. Although the emission wavelength is typically specified as a single wavelength, e.g., 670 nm, it will be understood by those of skill in the art that this specification refers to a wavelength range or emission filter band-pass that is centered on the specified wavelength. In some instances, light of the specified emission wavelength comprises light of the specified wavelength ±2 nm, ±5 nm, ±10 nm, ±20 nm, ±40 nm, ±80 nm, or more. In some instances, the emission wavelength used may or may not coincide with the emission peak maximum of the fluorescent indicator.

The terms “modification” or “modified” as used herein generally refer to any change in the structure, biological, chemical, or any composition thereof of a polymerase. For example, modifications may comprise a change in the amino acid sequence of the polymerase (e.g., in comparison with a reference polymerase). In some embodiments, modifications may comprise one or more changes in the amino acid sequence including: amino acid additions, deletions, and/or substitutions (e.g., including conservative and non-conservative substitutions).

As used herein, the term “conservative” refers to an amino acid mutation where an amino acid is substituted by another amino acid having highly similar properties. For example, one or more amino acids comprising nonpolar or aliphatic side chains (for example, glycine, alanine, valine, leucine, or isoleucine) can be substituted for each other. Similarly, one or more amino acids comprising polar, uncharged side chains (for example, serine, threonine, cysteine, methionine, asparagine or glutamine) can be substituted for each other. Similarly, one or more amino acids comprising aromatic side chains (for example, phenylalanine, tyrosine or tryptophan) can be substituted for each other. Similarly, one or more amino acids comprising positively charged side chains (for example, lysine, arginine or histidine) can be substituted for each other. Similarly, one or more amino acids comprising negatively charged side chains (for example, aspartic acid or glutamic acid) can be substituted for each other. In some embodiments, the modified polymerase is a variant that comprises one or more of these conservative amino acid substitutions, or any combination thereof.

As used herein, the terms “identical” or “percent identity,” when used with respect to two or more nucleic acid or polypeptide sequences, refer to two or more sequences that are the same or, alternatively, have a specified percentage of amino acid residues or nucleotides that are the same, when compared and aligned for maximum correspondence, as measured using any one or more of the following sequence comparison algorithms: Needleman-Wunsch (see, e.g., Needleman, Saul B.; and Wunsch, Christian D. (1970). “A general method applicable to the search for similarities in the amino acid sequence of two proteins” Journal of Molecular Biology 48 (3):443-53); Smith-Waterman (see, e.g., Smith, Temple F.; and Waterman, Michael S., “Identification of Common Molecular Subsequences” (1981) Journal of Molecular Biology 147:195-197); or BLAST (Basic Local Alignment Search Tool; see, e.g., Altschul S F, Gish W, Miller W, Myers E W, Lipman D J, “Basic local alignment search tool” (1990) J Mol Biol 215 (3):403-410).

As used herein, the terms “substantially identical” or “substantial identity” when used with respect to two or more nucleic acid or polypeptide sequences, refer to two or more sequences or subsequences (such as biologically active fragments) that have at least 60%, at least 70%, at least 75%, at least 80%, at least 85%, at least 90%, at least 95%, at least 96%, at least 97%, at least 98%, or at least 99% nucleotide or amino acid residue identity, when compared and aligned for maximum correspondence, as measured using a sequence comparison algorithm or by visual inspection. Substantially identical sequences are typically considered to be homologous without reference to actual ancestry. In some embodiments, “substantial identity” exists over a region of the sequences being compared. In some embodiments, substantial identity exists over a region of at least 25 residues in length, at least 50 residues in length, at least 100 residues in length, at least 150 residues in length, at least 200 residues in length, or greater than 200 residues in length. In some embodiments, the sequences being compared are substantially identical over the full length of the sequences being compared. Typically, substantially identical nucleic acid or protein sequences include less than 100% nucleotide or amino acid residue identity as such sequences would generally be considered “identical”.

Proteins and/or protein subsequences (such as biologically active fragments) are considered “homologous” when they are derived, naturally or artificially, from a common ancestral protein or protein sequence. Similarly, nucleic acids and/or nucleic acid sequences are considered homologous when they are derived, naturally or artificially, from a common ancestral amino acid sequence or nucleic acid sequence. Homology is generally inferred from sequence similarity between two or more nucleic acids or proteins (or biologically active fragments or sequences thereof). The precise percentage of similarity between sequences that is useful in establishing homology varies with the nucleic acid or protein at issue, but as little as 25% sequence similarity over 25, 50, 100, 150, or more nucleic acids or amino acid residues, is routinely used to establish homology. Higher levels of sequence similarity (e.g., 30%, 40%, 50%, 60%, 70%, 80%, 85%, 90%, 95%, 98% or 99%) can also be used to establish homology.

The section headings used herein are for organizational purposes only and are not to be construed as limiting the subject matter described.

### Nucleic Acid Polymerases

In some embodiments, the modified polymerase (or biologically active fragment thereof) includes one or more amino acid mutations that are located inside the catalytic domain of the modified polymerase, and wherein the polymerase has at least 80%, 85%, 90%, 95%, 96%, 97%, 98%, 99%, or more identity to any one of the modified polymerases disclosed herein. In some embodiments, the modified polymerase (or biologically active fragment thereof) includes one or more amino acid mutations that are located inside the catalytic domain of the modified polymerase, and wherein the polymerase has at least 80%, 85%, 90%, 95%, 96%, 97%, 98%, 99% or more identity to any one of SEQ ID NO: 1. In some embodiments, the modified polymerase (or biologically active fragment thereof) includes one or more amino acid mutations that are located inside the catalytic domain of the modified polymerase, and wherein the polymerase has at least 80%, 85%, 90%, 95%, 96%, 97%, 98%, 99% or more identity to any one of SEQ ID NO: 3. In some embodiments, the modified polymerase has at least 80%, 85%, 90%, 95%, 96%, 97%, 98%, 99% or more identity to one or more reference enzymes. For example, in some embodiments, one reference enzyme may be Bst and a second reference enzyme may be Mcu7. SEQ ID NO: 3 includes, for example Mcu7 DNA binding domain (SEQ ID NO: 4), an FL2 linker (GGGGSGGGGGS, SEQ ID NO: 12), a truncated Bst polymerase, and a C-terminal LF3 tag (leucine-glutamine).

In some embodiments, the modified polymerase (or biologically active fragment thereof) includes one or more amino acid mutations that are located inside the DNA binding domain of the polymerase. In some embodiments, the modified polymerase or biologically active fragment thereof can include at least 25, 50, 75, 100, 150, or more amino acid residues of the DNA binding domain of the modified polymerase. In some embodiments, the modified polymerase or biologically active fragment thereof can include any part of the DNA binding domain that comprises at least 25, 50, 75, 100, 150, or more contiguous amino acid residues. In some embodiments, the modified polymerase or biologically active fragment thereof can include at least 25 contiguous amino acid residues of the binding domain and can optionally include one or more amino acid residues at the C-terminal or the N-terminal that are outside of the binding domain. In some embodiments, the modified polymerase or a biologically active fragment can include any 25, 50, 75, 100, 150 or more contiguous amino acid residues of the binding domain coupled to any one or more non-binding domain amino acid residues. In some embodiments, the modified polymerase (or biologically active fragment thereof) includes one or more amino acid mutations that are located inside the DNA binding domain of the modified polymerase, where the polymerase has at least 80%, 85%, 90%, 95%, 96%, 97%, 98%, 99%, or more identity to any one of the modified polymerases disclosed herein. In some embodiments, the modified polymerase (or biologically active fragment thereof) includes one or more amino acid mutations that are located inside the DNA binding domain of the modified polymerase, where the polymerase has at least 80%, 85%, 90%, 95%, 96%, 97%, 98%, 99% or more identity to any one of SEQ ID NO: 1. In some embodiments, the modified polymerase (or biologically active fragment thereof) includes one or more amino acid mutations that are located inside the DNA binding domain of the modified polymerase, where the polymerase has at least 80%, 85%, 90%, 95%, 96%, 97%, 98%, 99% or more identity to any one of SEQ ID NO: 3.

In some embodiment, the polymerase includes a DNA binding domain. Exemplary DNA binding domains include Mcu7 DNA binding domain (SEQ ID NO: 4), SS07d DNA binding domains (SEQ ID NO: 5), Sac7d DNA binding domain (SEQ ID NO: 15), Sac7e DNA binding domain (SEQ ID NO: 16), 1XYI-A DNA binding domain (SEQ ID NO: 17), 1WTO-A DNA binding domain (SEQ ID NO: 18), DNA binding sequence of YP_009230321.1 (SEQ ID NO: 19), 5UFE-B DNA binding domain (SEQ ID NO: 20), 4CJ0-B DNA binding domain (SEQ ID NO: 21), DNA binding domain of WP_009071314.1 (SEQ ID NO: 22), 4CJ2-C DNA binding domain (SEQ ID NO: 23), or 2XIW-A DNA binding domain (SEQ ID NO: 24)

In some embodiments, the modified polymerase or a biologically active fragment thereof, includes one or more amino acid mutations that are located outside the catalytic domain (also referred to herein as the DNA binding cleft) of the polymerase. For example, the catalytic domains many polymerases known in the art have a shape that has been compared to a right hand and consists of “palm”, “thumb” and “finger” domains. The palm domain typically contains the catalytic site for the phosphoryl transfer reaction. The thumb is thought to play a role positioning the duplex DNA and in processivity and translocation. The fingers interact with the incoming nucleotide as well as the template base with which it is paired.

In some embodiments, the reference polymerase has or comprises the amino acid sequence of SEQ ID NO.: 1 or SEQ ID NO.:3, which do not include mutations from the BST polymerase. The modified polymerase has or comprises the amino acid sequence of the reference polymerase. In some embodiments, the modified polymerase comprises an amino acid sequence that is at least 80%, 85%, 90%, 91%, 92%, 93%, 94%, 95%, 96%, 97%, 98%, or 99% identical to the amino acid sequence of the reference polymerase. In some embodiments, the nucleic acid polymerase includes comprises a mutation, relative to SEQ ID NO: 1 or SEQ ID NO: 3, at an amino acid position selected from the group consisting of 495, 515, 526-534, 538, 549, 551-568, 570-590, 609-630, 635-637, 640, 655-663, 674, 681, 683, 688, 689, 701, 703-720, 728-730, 743, 761, 770-829, 872, and 873, wherein the amino acid position numbering is according to SEQ ID NO: 2. In some embodiments, the nucleic acid polymerase includes comprises a mutation, relative to SEQ ID NO: 1 or SEQ ID NO: 3, at an amino acid position selected from the group consisting of 495, 515, 529, 531, 538, 549, 558, 559, 561, 562, 563, 564, 565, 566, 567, 568, 572, 573, 575, 579, 580, 583, 622, 623, 628, 629, 630, 635, 636, 637, 640, 655, 660, 661, 662, 674, 681, 683, 688, 689, 701, 703, 704, 707, 710, 713, 716, 718, 743, 761, 780, 782, 785, 786, 788, 800, 806, 815, 819, 826, 827, 872, and 873, wherein the amino acid position numbering is according to SEQ ID NO: 2. In some embodiments, the modified polymerase further includes any one or more amino acid mutations selected from the group consisting of: V495F, E515K, N529R, P531Q, L538F, K549I, A558M, D559K, D559R, D559S, L561I, E562N, K563G, L564V, A565R, P566L, Y567H, H568P, E572D, N573E, L575I, Q579N, L580D, L580G, L583N, L583V, N622G, E623Y, I628L, I628M, R629A, L630D, L630P, K635S, I636M, I636L, R637M, F640V, S655N, R660C, V661I, L662M, A674V, I681V, T683K, T683V, D688M, I689F, I689Q, I689V, I689Y, M701L, R703A, R703H, R703P, Q704I, A707Y, F710Y, V713M, I716Q, D718A, D718K, F743R, F743Y, G761E, N780K, N782R, N782V, S785R, F786R, E788R, A800G, K806E, K806M, L815F, R819K, R819M, R819P, R819V, L826Y, Q827D, W872H, and Y873D, where the numbering is relative to the amino acid sequence of SEQ ID NO: 2. In some embodiments, the polymerase includes any one or more amino acid mutations listed in Table 4. Without being bound to any particular theory of operation, it can be observed that in some embodiments a modified polymerase including one or more of these mutations exhibits an altered (e.g., increased or decreased) processivity, or an altered (e.g., increased or decreased) rate of incorporation of nucleotides, or an altered (e.g., increased or decreased) rate of misincorporation of nucleotides.

### Fluorescence Resonance Energy Transfer Overview

As noted above, fluorescence resonance energy transfer (FRET) is a mechanism for non-radiative energy transfer between two light-absorbing molecules (e.g., fluorophores). Use of the FRET technique allows one to quantitatively detect molecular interactions over distances of tens of angstroms. The center-to-center distance between adjacent nucleotide pairs in double-stranded DNA is 3.4 nm (34 angstroms).

### Design of FRET-Base Polymerase Assays

A typical DNA polymerase reaction involves an oligonucleotide substrate (usually a hybrid of a bound primer and a template oligonucleotide, e.g., a DNA molecule), the DNA polymerase, and incoming deoxyribonucleoside triphosphates (dNTPs). In the course of the polymerase catalyzed reaction, the enzyme incorporates the incoming dNTPs into the extended primer molecule in a template-dependent manner. There are several different approaches by which the FRET technique can be utilized to detect nucleotide incorporation (or misincorporation). For example, in some instances, either the primer sequence or the template DNA molecule can be labeled with one member of the fluorescence probe pair (or FRET donor—acceptor pair)—either the fluorescence donor or the fluorescence acceptor—and the incoming dNTPs can be labeled with the other member. Any nucleotide incorporation (or misincorporation) event catalyzed by the polymerase is then detected by an increase in FRET efficiency between the two fluorophores (e.g., as a decrease in donor fluorescence or an increase in acceptor fluorescence). Alternatively, in some instances, both components (or members) of the FRET probe pair can be attached to the DNA template/primer complex, and the incoming nucleotides can be unlabeled. In this case, the successful incorporation (or misincorporation) of the incoming nucleotide(s) causes conformational changes of the primer/template complexes that result in measurable perturbation of the FRET efficiency. In yet another possible configuration, one of the FRET partner dyes can be attached to the polymerase, while the other can be attached to the template/primer hybrid or to the incoming dNTP. In this case, successful incorporation (or misincorporation) may cause a translocation of the labeled polymerase or bring the incoming labeled nucleotide in close proximity to the polymerase-bound dye, resulting in a measurable change in FRET efficiency.

Disclosed herein are homogenous polymerase assays designed to evaluate the ability of various DNA polymerases (or other polymerases, e.g., RNA polymerases, reverse transcriptases, and the like) to mis-incorporate labeled dNTPs into the growing primer molecule. Initial misincorporation assays were performed using an unlabeled primer molecule, a template oligonucleotide sequence labeled with a suitable FRET donor, and dNTPs labeled with a suitable FRET acceptor.

In one non-limiting example, the primer/template hybrid sequences listed in Table 1 were used to evaluate the ability of different polymerases to mis-incorporate dye-labeled dATPs.

TABLE 1
Non-limiting examples of paired primer/oligonucleotide template sequences.

| Sequence | Misincorporation | |
|---|---|---|
| Example No. | tested | Primer/oligo template pair |
| 1 | A opposite A | .....AGTCTGG 3′ |
| | | .....TCAGACCAGCTATTT 5′ (SEQ ID NO: 25) |
| 2 | G opposite T | .....AGTGCTG 3′ |
| | | TCACGACTGCTTT 5′ (SEQ ID NO: 26) |
| 3 | T opposite G | .....AGTCGGA 3′ |
| | | .....TCAGCCTGTTTTTT 5′ (SEQ ID NO: 27) |
| 4 | G opposite T | .....AGTCCCT 3′ |
| | | .....TCAGGGATTAAATCT 5′ (SEQ ID NO: 28) |

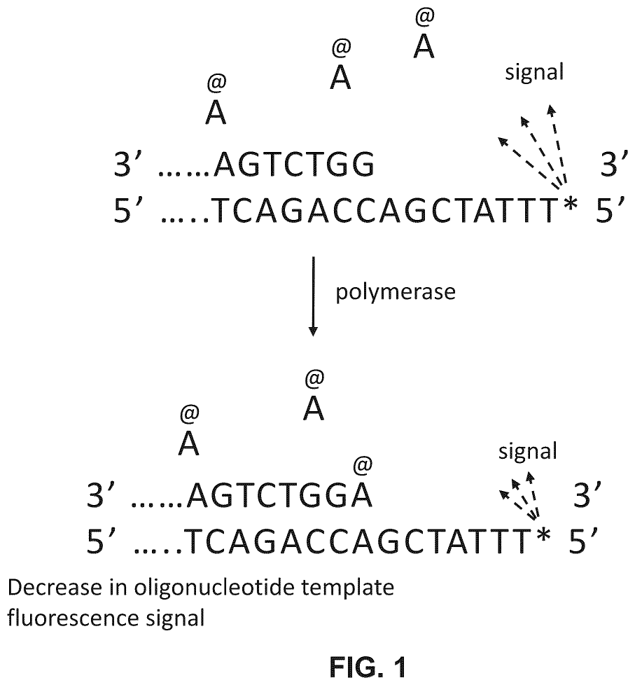

FIG. 1 illustrates the assay format using the first primer/template oligonucleotide sequence pair listed in Table 1. As illustrated in FIG. 1, the incoming labeled dNTPs used in this case were dATP-Atto633 (fluorescence acceptor; A@). The nucleotide misincorporation assay begins following the formation of a reaction mixture (FIG. 1, upper) comprising the template oligonucleotide sequence, primer sequence, the labeled non-complementary dNTPs, and the polymerase. Misincorporation of the fluorescence acceptor-labeled dATP is detectable by FRET, specifically through monitoring of the fluorescence intensity of the FRET donor (e.g., the labeled oligonucleotide) (FIG. 1, lower). The rate of decrease of the expected FRET signal is a measure of the efficiency of the polymerase being tested in mis-incorporating labeled dATPs (e.g., a decrease in the fluorescence intensity of the FRET donor indicates that more misincorporation events have occurred). In some instances, the fluorescence intensity of the fluorescence donor and/or acceptor may be monitored as a function of time following addition of the last component (e.g., the polymerase) to a reaction mixture comprising the template oligonucleotide sequence, the primer sequence, the labeled dNTPs, and the polymerase). In some instances, the fluorescence intensity of the fluorescence donor and/or acceptor may be monitored at a specified time (e.g., an endpoint) following addition of the last component (e.g., the polymerase) to a reaction mixture comprising the template oligonucleotide sequence, the primer sequence, the labeled dNTPs, and the polymerase). In some instances, a fluorescence donor—quencher pair may be used instead of a fluorescence donor-acceptor pair (e.g., with the fluorescence quencher attached to the dNTP comprising the complementary base to the template nucleotide at the 5′ end of the homopolymer or repeat sequence), and a decrease in the fluorescence intensity of the fluorescence donor is measured as a function of time (or at a specified endpoint) following addition of the final component to the assay reaction mixture. In some instances, the methods may be used to evaluate and compare different polymerases based on their respective abilities to mis-incorporate labeled nucleotides (e.g., with decreased ability to mis-incorporate being the desired attribute). In some instances, the methods may be used to evaluate, compare, and/or select a mutant polymerase from a library of mutant polymerases based on their respective abilities to mis-incorporate labeled nucleotides. In some instances, template sequences may comprise a sequence of oligonucleotide residues comprising two or more types of nucleotides. In some instances, the reaction mixture for misincorporation assays further comprises unlabeled complementary dNTPs. For example, for the template oligonucleotide in FIG. 1, unlabeled dTTPs may be added to the reaction mixture (e.g., to observe competition between the complementary nucleotide and the non-complementary nucleotide).

Further disclosed herein are homogeneous polymerase assays designed to evaluate the ability of various DNA polymerases (or other polymerases, e.g., RNA polymerases, reverse transcriptases, and the like) to sequentially incorporate labeled dNTPs into the growing primer molecule. Initial incorporation experiments were performed using a primer molecule labeled with a single donor and dNTPs labeled with a suitable acceptor. FRET theory predicts an increase of FRET efficiency with an increasing number of fluorescence acceptor moieties located within the effective FRET distance (e.g., approximately the Forster distance) of the fluorescence donor. In practice, however, it was difficult to differentiate between multiple sequential incorporation events (e.g., in a homopolymer sequence) using this initial assay format.

An alternative assay format, where both the fluorescence donor and the fluorescence acceptor are located on the incoming nucleotides (the other components of the assay system—the template DNA and the polymerase enzyme—remain unlabeled), provided improved ability to detect multiple sequential nucleotide incorporation events used dye-labeled dNTPs. The template oligonucleotide sequences (template DNA strands) used in the assay are designed to enable the incorporation of multiple fluorescence donor-labeled dNTPs first (e.g., in a homopolymer sequence), followed by eventual incorporation of a fluorescence acceptor-labeled dNTP molecule, whereupon a detectable FRET signal between the donor(s) and acceptor is registered. In a reaction mixture comprising a plurality of primed template oligonucleotides undergoing asynchronous polymerase binding and polymerase-catalyzed nucleotide incorporation, the eventual incorporation of the fluorescence acceptor-labeled dNTP molecule into the plurality of primer extension strands leads to an increase in acceptor fluorescence intensity as a function of time, where the rate of increase is proportional to an average incorporation rate for the labeled nucleotides (under conditions that polymerase binding to the primed template molecule is not rate-limiting).

In one non-limiting example, the primer/template hybrid sequences listed in Table 2 were used to evaluate the ability of different polymerases to sequentially incorporate one, two, three, or four dye-labeled dGTPs.

TABLE 2
Non-limiting examples of paired primer/oligonucleotide template sequences.

| Sequence | Incorporation | |
|---|---|---|
| Example No. | assayed | Primer/oligo template pair |
| 1 | 1 dGTP | .....AGGCT 3′ |
| | | .....TCCGACA 5′ |
| 2 | 2 dGTPs | .....AGGCT 3′ |
| | | .....TCCGACCA 5′ |
| 3 | 3 dGTPs | .....AGGCT 3′ |
| | | .....TCCGACCCA 5′ |
| 4 | 4 dGTPs | ....AGGCT 3′ |
| | | ....TCCGACCCCA 5′ (SEQ ID NO: 29) |
| 5 | 5 dGTPs | ....AGGCT 3′ |
| | | ....TCCGACCCCCA 5′ (SEQ ID NO: 30) |
| 6 | 3 dATPs | ....3′ |
| | | ....TTTACTTT 5′ |
| 7 | 3 dATPs | ....3′ |
| | | ....TTTGCTTTT 5′ |
| 8 | 3 dCTPs | ...CTG 3′ |
| | | ...GACGGGACTTT 5′ (SEQ ID NO: 31) |
| 9 | 3 dGTPs | ...CTG 3′ |
| | | ...GACCCCAGTTT 5′ (SEQ ID NO: 32) |

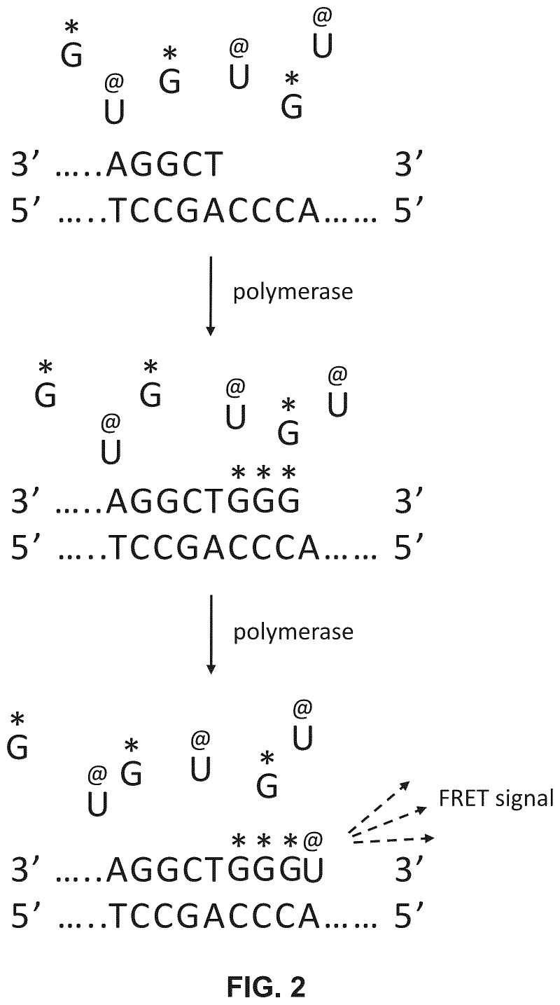

FIG. 2 illustrates the assay format using the third primer/template oligonucleotide sequence pair listed in Table 2. As illustrated in FIG. 2, the incoming labeled dNTPs used in this case were dGTP-Atto532 (fluorescence donor; G*) and dUTP-Atto633 (fluorescence acceptor; U@). The nucleotide incorporation reaction begins following the formation of a reaction mixture (FIG. 2, upper) comprising the template oligonucleotide sequence, primer sequence, the labeled complementary dNTPs, and the polymerase. Initially, only the dGTP derivative is incorporated by the polymerase (FIG. 2, middle). These initial incorporation events remain undetectable by FRET (e.g., there is no detectable acceptor fluorescence intensity). The subsequent incorporation of the fluorescence acceptor-labeled dUTP becomes possible only upon completion of the poly-G sequence. A FRET signal becomes detectable when the dUTP derivative is added after incorporation of the last dGTP (FIG. 2, lower). The rate of increase of the expected FRET signal is a measure of the efficiency of the polymerase being tested in incorporating multiple sequential labeled dNTPs. In some instances, by using template sequences comprising a different number of oligonucleotide residues of the same type (e.g., one, two, three, four, or more than four C residues), one may evaluate polymerase processivity (i.e., the average number of nucleotides incorporated by the polymerase per association event with the template strand) and/or determine a maximum effective length of a sequence (e.g., a homopolymer sequence or a repeat sequence) for which the polymerase is capable of sustaining continuous primer extension reactions using labeled nucleotides. In some instances, the fluorescence intensity of the fluorescence donor and/or acceptor may be monitored as a function of time following addition of the last component (e.g., the polymerase) to a reaction mixture comprising the template oligonucleotide sequence, the primer sequence, the labeled dNTPs, and the polymerase). In some instances, the fluorescence intensity of the fluorescence donor and/or acceptor may be monitored at a specified time (e.g., an endpoint) following addition of the last component (e.g., the polymerase) to a reaction mixture comprising the template oligonucleotide sequence, the primer sequence, the labeled dNTPs, and the polymerase). In some instances, a fluorescence donor—quencher pair may be used instead of a fluorescence donor—acceptor pair (e.g., with the fluorescence quencher attached to the dNTP comprising the complementary base to the template nucleotide at the 5′ end of the homopolymer or repeat sequence), and a decrease in the fluorescence intensity of the fluorescence donor is measured as a function of time (or at a specified endpoint) following addition of the final component to the assay reaction mixture. In some instances, the methods may be used to evaluate and compare different polymerases based on their respective abilities to incorporate labeled nucleotides or their respective processivities for, e.g., homopolymer sequences. In some instances, the methods may be used to evaluate, compare, and/or select a mutant polymerase from a library of mutant polymerases based on their respective abilities to incorporate labeled nucleotides or their respective processivities for, e.g., homopolymer sequences. In some instances, template sequences may comprise a sequence of oligonucleotide residues comprising two or more types of nucleotides. In some such instances, at least one type of nucleotides in the sequence of oligonucleotide residues must comprise the first member of the fluorescence (e.g., fluorescence donor-acceptor or fluorescence donor-quencher) probe pair, and the second nucleotide comprising the second member of the fluorescence probe pair must be a different type of nucleotide than nucleotides in the sequence of oligonucleotide residues.

In some instances, the methods comprise the use of three fluorophores (e.g., two fluorescence donor—acceptor pairs comprising a common member) to provide more information on the kinetics of the polymerase reaction (important, e.g., for sequencing reaction efficacy). In some instances, for example, a first fluorophore (e.g., a fluorescence donor) may be attached to a nucleotide residue at the 5′ end of the template oligonucleotide sequence, a second fluorophore which is a fluorescence acceptor for the first fluorophore (fluorescence donor) and is also a fluorescence donor for the third fluorophore is attached to the dNTPs that are complementary to the homopolymer or repeat sequence portion of the template oligonucleotide sequence, and a third fluorophore which is a fluorescence acceptor for the second fluorophore is attached to the dNTP that is complementary to the template nucleotide residue at the 5′ end of the homopolymer or repeat sequence portion of the template oligonucleotide sequence. In some instances, the fluorescence intensity of the first fluorophore, the second fluorophore, and/or the third fluorophore may be monitored as a function of time following addition of the last component (e.g., the polymerase) to a reaction mixture comprising the template oligonucleotide sequence, the primer sequence, the labeled dNTPs, and the polymerase). In some instances, the fluorescence intensity of the first fluorophore, the second fluorophore, and/or the third fluorophore may be monitored at a specified time (e.g., an endpoint) following addition of the last component (e.g., the polymerase) to a reaction mixture comprising the template oligonucleotide sequence, the primer sequence, the labeled dNTPs, and the polymerase). In some instances, a fluorescence quencher may be used instead of one of the fluorophores (e.g., instead of the third fluorophore), and a decrease in the fluorescence intensity of the first fluorophore or the second fluorophore is measured as a function of time (or at a specified endpoint) following addition of the final component to the assay reaction mixture. In some instances, the methods may be used to evaluate and compare different polymerases based on their respective abilities to incorporate labeled nucleotides or their respective processivities for, e.g., homopolymer sequences. In some instances, the methods may be used to evaluate, compare, and/or select a mutant polymerase from a library of mutant polymerases based on their respective abilities to incorporate labeled nucleotides or their respective processivities for, e.g., homopolymer sequences.

In some instances of any of the assay formats described herein, the methods may be used to calculate or estimate an average nucleotide incorporation rate by determining a rate of change of the detected FRET signal and making use of the known composition and length of the template oligonucleotide sequence (e.g., the number of nucleotide residues in the template oligonucleotide sequence). In some instances of any of the assay formats described herein, the methods may be used to determine an effective maximum homopolymer length for which the polymerase is capable of efficiently incorporating labeled complementary nucleotides, or to determine an ability of the polymerase to sustain continuous primer extension reactions using labeled nucleotides, by comparing FRET signal measurements made using template oligonucleotide sequences of different lengths (e.g., comprising different numbers of nucleotide residues in the template oligonucleotide sequence).

### Nucleotides

As noted above, the term “nucleotide” as used herein encompasses a nucleoside triphosphate, e.g., a deoxyribonucleoside triphosphate (dNTP) or ribonucleoside triphosphate (NTP) comprising: (i) a nitrogenous base (or nucleobase) (e.g., adenine (A), cytosine (C), guanine (G), thymine (T), or uracil (U)), (ii) a 5 carbon sugar moiety (either deoxyribose or ribose, respectively); and (iii) three phosphate moieties. In some instances, a nucleotide comprises a non-natural nucleotide that comprises a non-natural (or synthetic) nucleobase (see, for example, Walsh, J. and Beuning, P. (2012), “Synthetic Nucleotides as Probes of DNA Polymerase Specificity”, J. Nucleic Acids 2012:530963). Examples of non-natural nucleobases include, but are not limited to, isocytosine bases, isoguanosine bases, methyl-substituted phenyl nucleobase analog (e.g., monomethylated, dimethylated, trimethylated, or tetramethylated benzene analogs), hydrophobic nucleobase analogs (e.g., 7-propynyl isocarbostyril nucleoside (dPICS), isocarbostyril nucleoside (ICS), 3-methylnaphthalene (3MN), or azaindole (7AI)), purine/pyrimidine mimics (e.g., a substituted azole heterocyclic carboxamide or a substituted indole scaffold), or fluorescent base analogs (e.g., 2-aminopurine (2AP) or the cytosine analogs 1,3-Diaza-2-oxophenothiazine and 1,3-Diaza-2-oxophenoxazine). In some instances, a nucleotide may further comprise a label, tag, or dye (e.g., a fluorophore or quantum dot) attached directly to the nucleotide.

### Linkers:

In some instances, the labeled nucleotides of the present disclosure comprise a label, tag, or dye (e.g., a fluorophore or quantum dot) attached to the nucleotide via a linker moiety. In some instances, the use of a linker to attach the fluorophore or other optical label to a nucleotide may help to reduce quenching of the associated signal when performing, e.g., sequencing reactions. In some embodiments, a linker moiety comprises a cleavable moiety such as a disulfide group. In some embodiments, lengths of functional groups connecting the cleavable to the nucleotide and/or dye may vary. In some embodiments, a linker moiety further comprises a spacer moiety such as a polyhydroxyproline (hyp-n) group. Examples of suitable linker molecules include, but are not limited to, aminoethyl-SS-propionic acid (epSS), aminoethyl-SS-benzoic acid, aminohexyl-SS-propionic acid, hyp-10, and hyp-20. Labeled nucleotides comprising linkers are described in more detail in International Patent Application Publication No. WO 2020/172197, which is incorporated herein by reference in its entirety.

### Template Oligonucleotide Sequences

The template oligonucleotide sequences used in the disclosed methods comprise a primer binding site, one or more nucleotide residues (e.g., a single nucleotide residue, or one or more nucleotide residues comprising the same base, two or more consecutive nucleotide residues having the same base, or a mixture of different bases), and at least one nucleotide residue at a 5′ end of the one or more nucleotide residues that is different from the one or more nucleotide residues (e.g., at least one nucleotide residue comprising a different base from those present in the one or more nucleotide residues). In some instances, the template oligonucleotide sequence may further comprise flanking sequences of arbitrary length at either the 3′ and/or 5′ end of this basis template oligonucleotide sequence.

In some instances, the number of nucleotide residues in a sequence segment comprising the one or more nucleotide residues comprises 1, 2, 3, 4, 5, 6, 7, 8, 9, 10, or more than 10 nucleotide residues. In some instances, the one or more nucleotide residues are of the same type (e.g., have the same base) and comprise a homopolymer sequence (i.e., a continuous sequence of nucleotide residues having the same base).

In some instances, the one or more nucleotide residues may comprise, for example, a single nucleotide residue, a homopolymer sequence, a short tandem repeat sequence comprising a dinucleotide (e.g., AC), trinucleotide (e.g., AAC), a tetranucleotide (e.g., AACT), or longer repeat pattern for which the bases present in the one or more repeats differ. In some instances, for example, the one or more nucleotide residues may comprise a short tandem repeat sequence that has a repeat pattern of from 2 to 16 nucleotides in length and include from 2 to 10 repeats of the repeat pattern. In some instances, the one or more nucleotide residues may comprise a defined sequence, an arbitrary or random sequence, a partially-random sequence (e.g., a mix of defined and random subsequences), or non-random sequence of nucleotide residues ranging from 2 to 50 nucleotide residues in length.

In some instances, the overall length of the template oligonucleotide sequence, including the primer binding site, the one or more nucleotide residues, the at least one nucleotide residue at the 5′ end of the one or more nucleotide residues that is different than the one or more nucleotide residues, and any flanking sequences at either the 3′ end and/or the 5′ end of the template, may range from about 15 nucleotides in length to about 50 nucleotides in length. In some instances, the overall length of the template oligonucleotide sequence may be at least 15 nucleotides, at least 16 nucleotides, at least 17 nucleotides, at least 18 nucleotides, at least 19 nucleotides, at least 20 nucleotides, at least 25 nucleotides, at least 30 nucleotides, at least 35 nucleotides, at least 40 nucleotides, at least 45 nucleotides, or at least 50 nucleotides. In some instances, the overall length of the template oligonucleotide sequence may be at most 50 nucleotides, at most 45 nucleotides, at most 40 nucleotides, at most 35 nucleotides, at most 30 nucleotides, at most 25 nucleotides, at most 20 nucleotides, at most 19 nucleotides, at most 18 nucleotides, at most 17 nucleotides, at most 16 nucleotides, or at most 15 nucleotides. Any of the lower and upper values described in this paragraph may be combined to form a range included within the disclosure, for example, in some instances the overall length of the template oligonucleotide sequence may range from about 16 nucleotides to about 35 nucleotides. Those of skill in the art will recognize that the overall length of the template oligonucleotide sequence may have any value within this range, e.g., about 24 nucleotides.

In some instances, the template oligonucleotide sequence comprises a label, tag, or dye (e.g., a fluorophore or quantum dot) attached directly to a nucleotide residue of the template sequence (e.g., the 3′ or 5′ nucleotide residue of the template sequence). In some instances, the template oligonucleotide sequence comprises a label, tag, or dye (e.g., a fluorophore or quantum dot) attached to a nucleotide residue of the template sequence (e.g., the 3′ or 5′ nucleotide residue of the template sequence) via a linker moiety, as described elsewhere herein.

### Primer Sequences

The disclosed methods comprise the use of primer sequences, e.g., short oligonucleotide sequences that are complementary to and hybridize with the template oligonucleotide sequence at the primer binding site, thereby forming a double-stranded segment of nucleic acid that is recognized and bound by a polymerase to trigger incorporation of nucleotides at the 3′-OH terminus of the annealed primers. In some instances, the length of the primer sequence may range from about 8 nucleotides in length to about 30 nucleotides in length. In some instances, the length of the primer may be at least 8 nucleotides, at least 10 nucleotides, at least 12 nucleotides, at least 14 nucleotides, at least 16 nucleotides, at least 20 nucleotides, at least 22 nucleotides, at least 24 nucleotides, at least 26 nucleotides, at least 28 nucleotides, or at least 30 nucleotides. In some instances, the length of the primer may be at most 30 nucleotides, at most 28 nucleotides, at most 26 nucleotides, at most 24 nucleotides, at most 22 nucleotides, at most 20 nucleotides, at most 18 nucleotides, at most 16 nucleotides, at most 14 nucleotides, at most 12 nucleotides, at most 10 nucleotides, or at most 8 nucleotides. Any of the lower and upper values described in this paragraph may be combined to form a range included within the disclosure, for example, the length of the primer may range from about 10 nucleotides to about 22 nucleotides. Those of skill in the art will recognize that the length of the primer may have any value within this range, e.g., about 19 nucleotides.

### Fluorescence Resonance Energy (FRET) Probe Pairs

Any of a variety of fluorescence probe pairs (or FRET probe pairs) known to those of skill in the art may be used in implementing the methods. Examples of suitable fluorescence donor-acceptor pairs include, but are not limited to, FITC—Rhodamine, Alexa488—Cy3, Cy3—Cy5, Atto550-Atto647N, Alexa546—Alexa647, Pacific Blue-Atto532, or Atto532—Atto633. Examples of suitable fluorescence donor—quencher pairs include, but are not limited to, Quasar670—BHQ2, CalRed—BHQ2, Quasar570—BHQ2, TET—BHQ2, or TAMRA—BHQ2. In some preferred instances, the assay methods comprise the use of Atto 532, Atto633, and/or Pacific Blue.

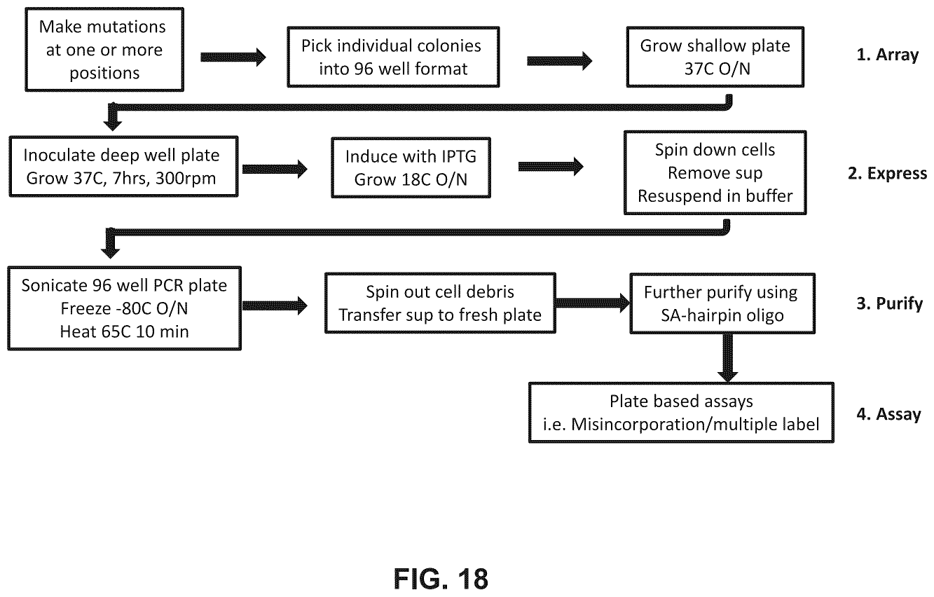

In some instances, the methods may be used to evaluate, rank, and/or select mutant polymerases from a library of mutant polymerases. FIG. 18 illustrates a non-limiting example workflow for generating and assaying a library of mutant polymerases. Libraries of mutated polymerases and other proteins may be produced using any of a variety of techniques known to those of skill in the art, and may utilize random mutagenesis, site-directed mutagenesis, combinatorial mutagenesis, or insertional mutagenesis. In one approach, for example, site-directed mutagenesis may be performed using a synthesized oligonucleotide primer containing the mutation as part of a primer extension reaction with DNA polymerase, followed by cloning and expression in the desired organism, and subsequent purification of the mutant polymerase protein (see, e.g., Hemsley, et al. (1989), “A Simple Method for Site-Directed Mutagenesis Using the Polymerase Chain Reaction”, Nucleic Acids Research 17(16):6545; and Kille, et al. (2013), “Reducing codon redundancy and screening effort of combinatorial protein libraries created by saturation mutagenesis”, ASC Synthetic Biology 2(83-92)).

After library preparation, nucleic acid sequencing can verify the introduction of the one or more amino acid mutations, e.g., by comparing a polymerase construct sequence against a standard or reference polymerase. Once verified, the construct containing the one or more of the amino acid mutations can be transformed into bacterial cells and expressed.

Typically, colonies containing mutant expression constructs are inoculated in media, induced, and grown to a desired optical density before being harvested (often via centrifugation). Spun down cells may be resuspended in a buffer solution. The harvested cells may be lysed via any means known in the art (e.g., physical, mechanical, or chemical lysing). For example, cells may be sonicated, heated, frozen, exposed to detergents, etc. or any combination thereof. Cellular debris from lysed cells may be extracted (e.g., via centrifugation). Mutant expression constructs may be purified from the supernatant (e.g., resulting from the centrifugation). It will be readily apparent to one skilled in the art that the supernatant can be purified by any suitable means and/or via any number of steps (e.g., depending on the purity of construct required and/or various chemical properties of a construct). Typically, a column for analytical or preparative protein purification is selected. In some embodiments, a modified polymerase or biologically active fragment thereof prepared using the methods described herein can be purified, without limitation, over a heparin column.

Once purified, the modified polymerase or biologically active fragment thereof can be assessed using any suitable method for various polymerase activities, with the polymerase activity being assessed depending on the application of interest. For example, to obtain a polymerase construct for use in amplifying or sequencing a nucleic acid molecule of about 400 bp in length, assays may include determination of polymerase activities such as increased processivity and/or increased dissociation time constant relative to a reference polymerase. In another example, an application requiring deep targeted-resequencing of a nucleic acid molecule of about 100 bp in length may include a polymerase with increased proofreading activity or increased minimum read length. In some embodiments, the one or more polymerase activities assessed can be related to polymerase performance or polymerase activity of a reference polymerase. In some embodiments, a modified polymerase or biologically active fragment thereof prepared according to the methods described herein can be assessed for nucleotide incorporation, processivity, nucleotide misincorporation, etc.

In some embodiments, a modified polymerase or biologically active fragment thereof can be assessed individually with respect to known values in the art for an analogous polymerase. In some embodiments, a modified polymerase or biologically active fragment thereof prepared according to the methods can be assessed against a known or reference polymerase under similar or identical conditions.

### Other Assay Reaction Components:

In some instances, the assay reaction mixture used in the methods described herein comprises a variety of additional assay components. Examples include, but are not limited to, pH buffers, salts, monovalent ions, divalent ions, zwitterions, detergents and surfactants, coenzymes, inorganic or organic cofactors, and the like.

### Fluorescence Detection Instrumentation

In some instances, the methods may be performed using any of a variety of commercial fluorescence spectrophotometers, fluorometers, and/or microplate readers configured to detect fluorescence. In some instances, the methods may be performed using a custom-built fluorescence detection instrument.

Examples of commercial fluorometers that may be suitable for use in performing the methods include, but are not limited to, Molecular Devices SpectraMax fluorescence microplate readers (Molecular Devices, San Jose, CA), the Duetta fluorescence and absorbance spectrometer (Horiba, Piscataway, NJ), the Qubit 4 Fluorometer and NanoDrop 3300 Fluorospectrometer (ThermoFisher Scientific, Waltham, MA), the Quantus™ (Promega Corp., Madison, WI).

Examples of commercial microplate readers configured to detect fluorescence include, but are not limited to, the GloMax® Plate Reader (Promega Corp., Madison, WI), the Synergy LX Multi-Mode Reader (BioTek Instruments, Winooski, VT), and the Spark® and Infinite® series of multimode fluorescence plate readers (Tecan, Baldwin park, CA).

In some instances, the methods may be performed using a custom-built fluorescence detection instrument comprising one or more light sources, monochromators, diffraction gratings, slits, apertures, lenses, mirrors, dichroic reflectors, dichroic filters, band-pass filters, long-pass filters, short-pass filters, interference filters, detectors (e.g., photomultipliers (PMTs), avalanche photodiodes, charge-coupled devices (CCDs), CMOS sensors, etc.), cuvette holders, flow cell holders, light tight housings, or any combination thereof.

In some instances, the light source(s) of the fluorescence detection instrument, alone or in combination with one or more optical components, e.g., excitation optical filters and/or dichroic beam splitters, may produce excitation light at about 350 nm, 375 nm, 400 nm, 425 nm, 450 nm, 475 nm, 500 nm, 525 nm, 550 m, 575 nm, 600 nm, 625 nm, 650 nm, 675 nm, 700 nm, 725 nm, 750 nm, 775 nm, 800 nm, 825 nm, 850 nm, 875 nm, or 900 nm. Those of skill in the art will recognize that the excitation wavelength may have any value within this range of about 350-900 nm, e.g., about 620 nm.

In some instances, the light source(s) of the fluorescence detection instrument, alone or in combination with one or more optical components, e.g., excitation optical filters and/or dichroic beam splitters, may produce light at the specified excitation wavelength within a bandwidth of ±2 nm, ±5 nm, ±10 nm, ±20 nm, ±40 nm, ±80 nm, or greater. Those of skill in the art will recognize that the excitation bandwidths may have any value within this range, e.g., about ±18 nm.

In some instances, one or more detection channels of the fluorescence detection instrument comprise one or more optical components, e.g., emission optical filters and/or dichroic beam splitters, configured to collect emission light at about 350 nm, 375 nm, 400 nm, 425 nm, 450 nm, 475 nm, 500 nm, 525 nm, 550 m, 575 nm, 600 nm, 625 nm, 650 nm, 675 nm, 700 nm, 725 nm, 750 nm, 775 nm, 800 nm, 825 nm, 850 nm, 875 nm, or 900 nm. Those of skill in the art will recognize that the emission wavelength may have any value within this range of about 350-900 nm, e.g., about 825 nm.

In some instances, one or more detection channels of the fluorescence detection instrument comprise one or more optical components, e.g., emission optical filters and/or dichroic beam splitters, configured to collect light at the specified emission wavelength within a bandwidth of ±2 nm, ±5 nm, ±10 nm, ±20 nm, ±40 nm, ±80 nm, or greater, and direct it to one or more detector(s). Those of skill in the art will recognize that the excitation bandwidths may have any value within this range of ±2 nm to ±80 nm, e.g., about ±18 nm.

In some instances, the methods comprise the use of a fluorescence detection instrument configured for dual wavelength excitation and/or dual wavelength emission.

### Polymerase Library Screening Methods & Throughput

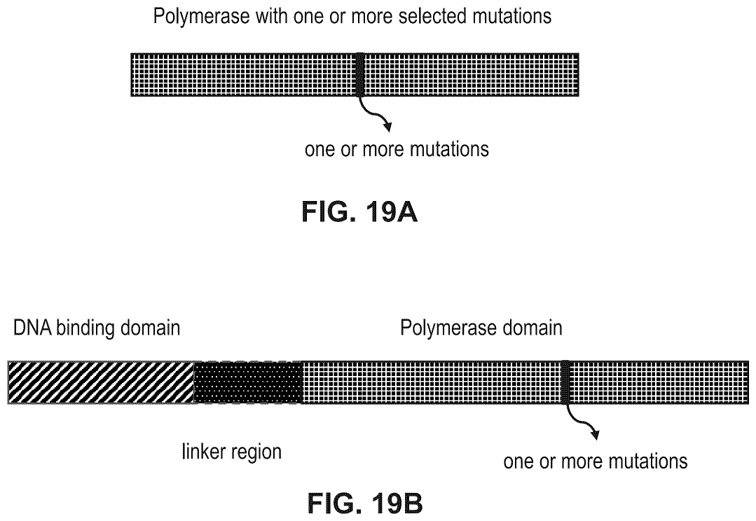

In some instances, a mutant polymerase may be selected, for example, if it is more efficient than a corresponding wild-type polymerase at incorporating nucleotides (e.g., labeled nucleotides) into a growing nucleic acid strand. In some instances, a mutant polymerase may be selected, for example, if it is less likely than a corresponding wild-type polymerase to mis-incorporate nucleotides (e.g., labeled nucleotides) during nucleic acid sequencing. As an example, BST DNA polymerase from Bacillus stearothermophilus is used in used to construct mutant polymerase library. In some embodiments, the BST DNA polymerase is a fragment of the BST DNA polymerase (e.g., SEQ ID NO.:1). In some embodiments, the BST DNA polymerase is the full-length enzyme (e.g., SEQ ID NO.:2). In some embodiments, the BST DNA polymerase fragment does not include the exonuclease domain from the full-length enzyme (e.g., FIG. 19A). In some embodiments, a BST DNA polymerase fragment is part of a hybrid protein where the polymerase domain is connected to a DNA binding domain via an optional linker region, as illustrated in FIG. 19B. An example of a hybrid polymerase protein can be found in SEQ ID NO.:3. In such embodiments, the DNA binding domain is a double stranded DNA binding domain such as an Mcu7 DNA binding domain (e.g., SEQ ID NO.: 4) or an SSO7d domain (e.g., SEQ ID NO.: 5). Additional DNA binding domains include but are not limited to any sequence of SEQ ID NO.:105 through SEQ ID NO.:114, or a homolog thereof. Other linkers may be used between the DNA binding domain and the polymerase domain, such as the FL2 linker (SEQ ID NO: 12), a native linker (SEQ ID NO: 13), or the SSO4 linker (SEQ ID NO: 14).

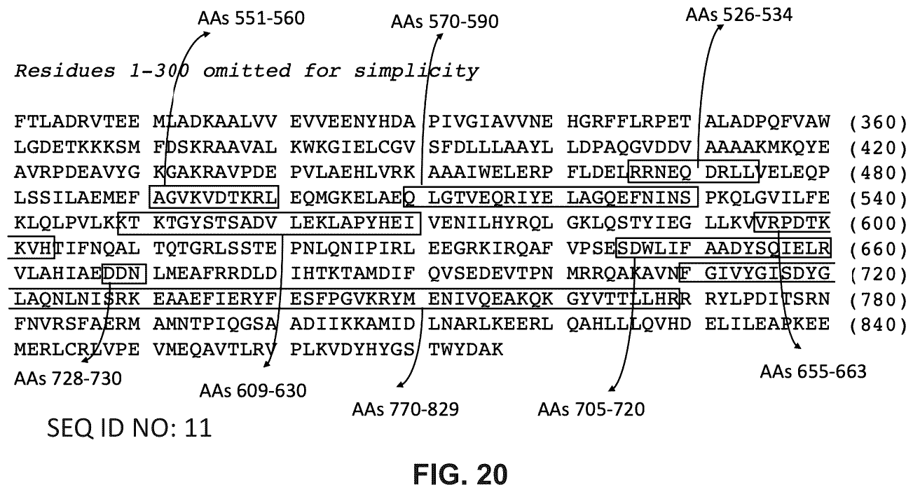

In some embodiments, amino acid residues in a polymerase that may directly interact with a nucleic substrate are subject to mutagenesis. For example, amino acid residues located within a certain distance (e.g., 5, 10, or 15 Angstroms) from a nucleic substrate can be subject to mutagenesis analysis. For example, FIG. 20 illustrates such amino acid positions in full length BST DNA polymerase (omitting residues 1-300 and showing only the remaining residues, SEQ ID NO.:11). In some embodiments, the mutant polymerase includes a single amino acid mutation. In some embodiments, the mutant polymerase includes two amino acid mutations, three amino acid mutations, four amino acid mutations, five amino acid mutations, six amino acid mutations, seven amino acid mutations, eight amino acid mutations, nine amino acid mutations, ten or more amino acid mutations. In some embodiments, amino acids located outside the pre-set distance (e.g., 5, 10, or 15 Angstroms) can also be subject to mutagenesis. In some embodiments, amino acids located within and outside the pre-set distance (e.g., 5, 10, or 15 Angstroms) can also be subject to mutagenesis.

### Nucleic Acid Substrates

As disclosed herein, any suitable nucleotide-linker-dye conjugates can be used as substrate in the activities assay, including but not limited to those disclosed in disclosed International Publication No. WO 2020/172197, which is hereby incorporated by reference in its entirety.

### Sequencing

Nucleic acid molecules may be sequenced using any suitable sequencing method to obtain sequencing data from the nucleic acid molecules, for example using a nucleic acid polymerase described herein. In some embodiments, the nucleic acid molecules comprise a sequencing adaptor sequence, wherein the sequencing adaptor sequence comprises a sequencing primer hybridization sequence. The sequencing primer may hybridize with the sequencing primer hybridization sequence of the sequencing adaptor sequence on the nucleic acid molecule and can be used to sequence the nucleic acid molecule, thus generating sequencing data.

Exemplary sequencing methods can include, but are not limited to, high-throughput sequencing, next-generation sequencing, sequencing-by-synthesis, flow sequencing, massively-parallel sequencing, shotgun sequencing, single-molecule sequencing, nanopore sequencing, pyrosequencing, semiconductor sequencing, sequencing-by-ligation, sequencing-by-hybridization, RNA-Seq, digital gene expression, single molecule sequencing by synthesis (SMSS), clonal single molecule array, sequencing by ligation, and Maxim-Gilbert sequencing. In some embodiments, the nucleic acid molecules may be sequenced using a high-throughput sequencer, such as an Illumina HiSeq2500, Illumina HiSeq3000, Illumina HiSeq4000, Illumina HiSeqX, Roche 454, Life Technologies Ion Proton, or open sequencing platform as described in U.S. Pat. No. 10,267,790, which is incorporated herein by reference in its entirety.

Other methods of sequencing and sequencing systems are known in the art. In some embodiments, the nucleic acid molecules are sequenced using a sequencing-by synthesis (SBS) method. In some embodiments, the nucleic acid molecules are sequenced using a “natural sequencing-by-synthesis” or “non-terminated sequencing-by-synthesis” method (see U.S. Pat. No. 8,772,473, which is incorporated herein by reference in its entirety).

Sequencing data associated with target nucleic acid molecules can be generated using a flow sequencing method that includes extending a primer bound to a template polynucleotide molecule according to a pre-determined flow cycle where, in any given flow position, a single type of nucleotide is accessible to the extending primer. In some implementations, two or more (e.g., two or three) different nucleotides may be simultaneously used in the same flow. In some embodiments, the nucleic acid molecules are sequenced using a plurality of sequencing flow steps, each sequencing flow step comprising combining the nucleic acid molecules hybridized to the sequencing primers with nucleotides, wherein at least a portion of the nucleotides are labeled (e.g., with a fluorescent label), and detecting the presence or absence of an incorporated nucleotide. In some instances, at least some of the nucleotides of the particular type include a label, which upon incorporation of the labeled nucleotides into the extending primer renders a detectable signal. In some instances, the at least of portion of the nucleotides is less than all of the nucleotides in each sequencing flow step. The resulting sequence by which such nucleotides are incorporated into the extended primer should be the reverse complement of the sequence of the template polynucleotide molecule. In some instances, for example, sequencing data is generated using a flow sequencing method that includes extending a primer using labeled nucleotides and detecting the presence or absence of a labeled nucleotide incorporated into the extending primer. While the following description is provided in reference to flow sequencing methods, it is understood that other sequencing methods may be used to sequence all or a portion of the sequenced region.

Flow sequencing includes the use of nucleotides to extend the primer hybridized to the polynucleotide. Nucleotides of a given base type (e.g., A, C, G, T, U, etc.) can be mixed with hybridized templates to extend the primer if a complementary base is present in the template strand. In some embodiments, the nucleotides in each sequencing flow step comprise nucleotides of a same base type. The nucleotides may be, for example, non-terminating nucleotides. When the nucleotides are non-terminating, more than one consecutive base can be incorporated into the extending primer strand if more than one consecutive complementary base is present in the template strand. The non-terminating nucleotides contrast with nucleotides having 3′ reversible terminators, wherein a blocking group is generally removed before a successive nucleotide is attached. If no complementary base is present in the template strand, primer extension ceases until a nucleotide that is complementary to the next base in the template strand is introduced. At least a portion of the nucleotides can be labeled so that incorporation can be detected. Most commonly, only a single nucleotide type is introduced at a time (i.e., discretely added), although two or three different types of nucleotides may be simultaneously introduced in certain embodiments. This methodology can be contrasted with sequencing methods that use a reversible terminator, wherein primer extension is stopped after extension of every single base before the terminator is reversed to allow incorporation of the next succeeding base.

The nucleotides can be introduced at a flow order during the course of primer extension, which may be further divided into flow cycles. The flow cycles are a repeated order of nucleotide flows and may be of any length. Nucleotides are added stepwise, which allows incorporation of the added nucleotide to the end of the sequencing primer of a complementary base in the template strand is present. Solely by way of example, the flow order of a flow cycle may be A-T-G-C, or the flow cycle order may be A-T-C-G. Alternative orders may be readily contemplated by one skilled in the art. The flow cycle order may be of any length, although flow cycles containing four unique base type (A, T, C, and G in any order) are most common. In some embodiments, the flow cycle includes 5, 6, 7, 8, 9, 10, 11, 12, 13, 14, 15, 16, 17, 18, 19, 20 or more separate nucleotide flows in the flow cycle order. Solely by way of example, the flow cycle order may be T-C-A-C-G-A-T-G-C-A-T-G-C-T-A-G, with these 16 separately provided nucleotides provided in this flow-cycle order for several cycles. Between the introductions of different nucleotides, unincorporated nucleotides may be removed, for example by washing the sequencing platform with a wash fluid.

The introduced nucleotides can include labeled nucleotides when determining the sequence of the template strand, and the presence or absence of an incorporated labeled nucleic acid can be detected to determine a sequence. The label may be, for example, an optically active label (e.g., a fluorescent label) or a radioactive label, and a signal emitted by or altered by the label can be detected using a detector. The presence or absence of a labeled nucleotide incorporated into a primer hybridized to a template polynucleotide can be detected, which allows for the determination of the sequence (for example, by generating a flowgram). In some embodiments, the labeled nucleotides are labeled with a fluorescent, luminescent, or other light-emitting moiety. In some embodiments, the label is attached to the nucleotide via a linker. In some embodiments, the linker is cleavable, e.g., through a photochemical or chemical cleavage reaction. For example, the label may be cleaved after detection and before incorporation of the successive nucleotide(s). In some embodiments, the label (or linker) is attached to the nucleotide base, or to another site on the nucleotide that does not interfere with elongation of the nascent strand of DNA. In some embodiments, the linker comprises a disulfide or PEG-containing moiety.

In some embodiments, the nucleotides introduced include only unlabeled nucleotides, and in some embodiments the nucleotides include a mixture of labeled and unlabeled nucleotides. For example, in some embodiments, the portion of labeled nucleotides compared to total nucleotides is about 90% or less, about 80% or less, about 70% or less, about 60% or less, about 50% or less, about 40% or less, about 30% or less, about 20% or less, about 10% or less, about 5% or less, about 4% or less, about 3% or less, about 2.5% or less, about 2% or less, about 1.5% or less, about 1% or less, about 0.5% or less, about 0.25% or less, about 0.1% or less, about 0.05% or less, about 0.025% or less, or about 0.01% or less. In some embodiments, the portion of labeled nucleotides compared to total nucleotides is about 100%, about 95% or more, about 90% or more, about 80% or more about 70% or more, about 60% or more, about 50% or more, about 40% or more, about 30% or more, about 20% or more, about 10% or more, about 5% or more, about 4% or more, about 3% or more, about 2.5% or more, about 2% or more, about 1.5% or more, about 1% or more, about 0.5% or more, about 0.25% or more, about 0.1% or more, about 0.05% or more, about 0.025% or more, or about 0.01% or more. In some embodiments, the portion of labeled nucleotides compared to total nucleotides is about 0.01% to about 100%, such as about 0.01% to about 0.025%, about 0.025% to about 0.05%, about 0.05% to about 0.1%, about 0.1% to about 0.25%, about 0.25% to about 0.5%, about 0.5% to about 1%, about 1% to about 1.5%, about 1.5% to about 2%, about 2% to about 2.5%, about 2.5% to about 3%, about 3% to about 4%, about 4% to about 5%, about 5% to about 10%, about 10% to about 20%, about 20% to about 30%, about 30% to about 40%, about 40% to about 50%, about 50% to about 60%, about 60% to about 70%, about 70% to about 80%, about 80% to about 90%, about 90% to less than 100%, or about 90% to about 100%.

Prior to generating the sequencing data, the polynucleotide is hybridized to a sequencing primer to generate a hybridized template. The polynucleotide may be ligated to an adapter during sequencing library preparation. The adapter can include a hybridization sequence that hybridizes to the sequencing primer. For example, the hybridization sequence of the adapter may be a uniform sequence across a plurality of different polynucleotides, and the sequencing primer may be a uniform sequencing primer. This allows for multiplexed sequencing of different polynucleotides in a sequencing library.

The polynucleotide may be attached to a surface (such as a solid support) for sequencing. The polynucleotides may be amplified (for example, by bridge amplification or other amplification techniques) to generate polynucleotide sequencing colonies. The amplified polynucleotides within the cluster are substantially identical or complementary (some errors may be introduced during the amplification process such that a portion of the polynucleotides may not necessarily be identical to the original polynucleotide). Colony formation allows for signal amplification so that the detector can accurately detect incorporation of labeled nucleotides for each colony. In some cases, the colony is formed on a bead using emulsion PCR and the beads are distributed over a sequencing surface. Examples for systems and methods for sequencing can be found in U.S. patent Ser. No. 10,344,328, which is incorporated herein by reference in its entirety.

The primer hybridized to the polynucleotide is extended through the nucleic acid molecule using the separate nucleotide flows according to the flow order (which may be cyclical according to a flow-cycle order), and incorporation of a nucleotide can be detected as described above, thereby generating the sequencing data set for the nucleic acid molecule.

Primer extension using flow sequencing allows for long-range sequencing on the order of hundreds or even thousands of bases in length. The number of flow steps or cycles can be increased or decreased to obtain the desired sequencing length. Extension of the primer can include one or more flow steps for stepwise extension of the primer using nucleotides having one or more different base types. In some embodiments, extension of the primer includes between 1 and about 1000 flow steps, such as between 1 and about 10 flow steps, between about 10 and about 20 flow steps, between about 20 and about 50 flow steps, between about 50 and about 100 flow steps, between about 100 and about 250 flow steps, between about 250 and about 500 flow steps, or between about 500 and about 1000 flow steps. The flow steps may be segmented into identical or different flow cycles. The number of bases incorporated into the primer depends on the sequence of the sequenced region, and the flow order used to extend the primer. In some embodiments, the sequenced region is about 1 base to about 4000 bases in length, such as about 1 base to about 10 bases in length, about 10 bases to about 20 bases in length, about 20 bases to about 50 bases in length, about 50 bases to about 100 bases in length, about 100 bases to about 250 bases in length, about 250 bases to about 500 bases in length, about 500 bases to about 1000 bases in length, about 1000 bases to about 2000 bases in length, or about 2000 bases to about 4000 bases in length.

Sequencing data can be generated based on the detection of an incorporated nucleotide and the order of nucleotide introduction. Take, for example, the flowing extended sequences (i.e., each reverse complement of a corresponding template sequence): CTG, CAG, CCG, CGT, and CAT (assuming no preceding sequence or subsequent sequence subjected to the sequencing method), and a repeating flow cycle of T-A-C-G (that is, sequential addition of T, A, C, and G nucleotides in repeating cycles). A particular type of nucleotides at a given flow position would be incorporated into the primer only if a complementary base is present in the template polynucleotide. An exemplary resulting flowgram is shown in Table 3, where 1 indicates incorporation of an introduced nucleotide and 0 indicates no incorporation of an introduced nucleotide. The flowgram can be used to derive the sequence of the template strand. For example, the sequencing data (e.g., flowgram) discussed herein represent the sequence of the extended primer strand, and the reverse complement of which can readily be determined to represent the sequence of the template strand. An asterisk (*) in Table 3 indicates that a signal may be present in the sequencing data if additional nucleotides are incorporated in the extended sequencing strand (e.g., a longer template strand).

TABLE 3
Exemplary Sequencing Data

| | Cycle 1 | | | | Cycle 2 | | | | Cycle 3 | | | |
|---|---|---|---|---|---|---|---|---|---|---|---|---|
| Flow Position | 1 | 2 | 3 | 4 | 5 | 6 | 7 | 8 | 9 | 10 | 11 | 12 |
| Base in Flow | T | A | C | G | T | A | C | G | T | A | C | G |
| Extended sequence: CTG | 0 | 0 | 1 | 0 | 1 | 0 | 0 | 1 | * | * | * | * |
| Extended sequence: CAG | 0 | 0 | 1 | 0 | 0 | 1 | 0 | 1 | * | * | * | * |
| Extended sequence: CCG | 0 | 0 | 2 | 1 | * | * | * | * | * | * | * | * |
| Extended sequence: CGT | 0 | 0 | 1 | 1 | 1 | * | * | * | * | * | * | * |
| Extended sequence: CAT | 0 | 0 | 1 | 0 | 0 | 1 | 0 | 0 | 1 | * | * | * |

The flowgram may be binary or non-binary. A binary flowgram detects the presence (1) or absence (0) of an incorporated nucleotide. A non-binary flowgram can more quantitatively determine a number of incorporated nucleotides from each stepwise introduction. For example, an extended sequence of CCG would include incorporation of two C bases in the extending primer within the same C flow (e.g., at flow position 3), and signals emitted by the labeled base would have an intensity greater than an intensity level corresponding to a single base incorporation. This is shown in Table 3. The non-binary flowgram also indicates the presence or absence of the base, and can provide additional information including the number of bases likely incorporated into each extending primer at the given flow position. The values do not need to be integers. In some cases, the values can be reflective of uncertainty and/or probabilities of a number of bases being incorporated at a given flow position.

In some embodiments, the sequencing data set includes flow signals representing a base count indicative of the number of bases in the sequenced nucleic acid molecule that are incorporated at each flow position. For example, as shown in Table 3, the primer extended with a CTG sequence using a T-A-C-G flow cycle order has a value of 1 at position 3, indicating a base count of 1 at that position (the 1 base being C, which is complementary to a G in the sequenced template strand). Also in Table 3, the primer extended with a CCG sequence using the T-A-C-G flow cycle order has a value of 2 at position 3, indicating a base count of 2 at that position for the extending primer during this flow position. Here, the 2 bases refer to the C-C sequence at the start of the CCG sequence in the extending primer sequence, and which is complementary to a G-G sequence in the template strand.

The flow signals in the sequencing data set may include one or more statistical parameters indicative of a likelihood or confidence interval for one or more base counts at each flow position. In some embodiments, the flow signal is determined from an analog signal that is detected during the sequencing process, such as a fluorescent signal of the one or more bases incorporated into the sequencing primer during sequencing. In some cases, the analog signal can be processed to generate the statistical parameter. For example, a machine-learning algorithm can be used to correct for context effects of the analog sequencing signal as described in published International patent application WO 2019084158 A1, which is incorporated by reference herein in its entirety. Although an integer number of zero or more bases are incorporated at any given flow position, a given analog signal many not perfectly match with the analog signal. Therefore, given the detected signal, a statistical parameter indicative of the likelihood of a number of bases incorporated at the flow position can be determined. Solely by way of example, for the CCG sequence in Table 3, the likelihood that the flow signal indicates 2 bases incorporated at flow position 3 may be 0.999, and the likelihood that the flow signal indicates 1 base incorporated at flow position 3 may be 0.001. The sequencing data set may be formatted as a sparse matrix, with a flow signal including a statistical parameter indicative of a likelihood for a plurality of base counts at each flow position.

Additional details regarding exemplary flow sequencing methods for use with the methods described herein includes the flow sequencing described in US 2020/0392584 A1, US 2020/0377937 A1, and US 2020/0372971 A1, which are incorporated herein by reference in their entirety.

### Exemplary Embodiments

The following embodiments are exemplary and are not intended to limit the scope of the claims.

Embodiment 1. A nucleic acid polymerase, comprising an amino acid sequence having at least 95% sequence identity to SEQ ID NO:1, wherein the polymerase comprises a mutation, relative to SEQ ID NO: 1, at an amino acid position selected from the group consisting of 526-534, 551-560, 570-590, 609-630, 655-663, 705-720, 728-730, and 770-829, wherein the amino acid position numbering is according to SEQ ID NO: 2.

Embodiment 2. The polymerase of embodiment 1, wherein the mutation is at amino acid position 360, 531, 538, 558, 566, 567, 568, 573, 579, 580, 583, 622, 628,629, 630, 635, 636, 637, 640, 716, 718, 782, 785, 786, 806, 815, 819, 826, or 827.

Embodiment 3. The polymerase of embodiment 2, wherein the mutation is L360P, P531Q, L538F, A558M, P566L, Y567H, H568P, N573E, Q579N, L580D, L580G, L583N, L583V, N622G, I628L, I628M, R629A, L630D, L630P, K635S, I636M, I636L, R637M, F640V, I716Q, D718K, N782R, S785R, F786R, K806E, K806M, L815F, R819K, R819M, R819P, R819V, L826Y or Q827D.

Embodiment 4. The polymerase of embodiment 1, wherein the polymerase comprises at least two mutations, relative to SEQ ID NO: 1, at amino acid positions selected from the group consisting of 526-534, 551-560, 570-590, 609-630, 655-663, 705-720, 728-730, and 770-829.

Embodiment 5. The polymerase of embodiment 4, wherein the at least two mutations are at amino acid positions 360, 531, 538, 558, 566, 567, 568, 573, 579, 580, 583, 622, 628, 629, 630, 635, 636, 637, 640, 716, 718, 782, 785, 786, 806, 815, 819, 826, or 827.

Embodiment 6. The polymerase of embodiment 5, wherein the at least two mutations comprise two or more of L360P, P531Q, L538F, A558M, P566L, Y567H, H568P, N573E, Q579N, L580D, L580G, L583N, L583V, N622G, I628L, I628M, R629A, L630D, L630P, K635S, I636M, I636L, R637M, F640V, I716Q, D718K, N782R, S785R, F786R, K806E, K806M, L815F, R819K, R819M, R819P, R819V, L826Y or Q827D.

Embodiment 7. The polymerase of any one of embodiments 1-6, wherein the amino acid sequence comprises at least 95% identity to SEQ ID NO: 2.

Embodiment 8. The polymerase of any one of embodiments 1-6, wherein the amino acid sequence further comprises a DNA binding domain.

Embodiment 9. The polymerase of embodiment 8, wherein the DNA binding domain comprises a sequence at least 95% identical to SEQ ID NO: 4 or SEQ ID NO: 5.

Embodiment 10. The polymerase of embodiment 8, wherein the DNA binding domain comprises a sequence of SEQ ID NO: 4 or SEQ ID NO: 5.

Embodiment 11. The polymerase of any one of embodiments 8-10, wherein the DNA binding domain is attached to the N-terminus of a polymerase domain.

Embodiment 12. The polymerase of embodiment 11, wherein the DNA binding domain is attached to the N-terminus of the polymerase domain through an amino acid linker.

Embodiment 13. A nucleic acid polymerase, comprising an amino acid sequence having at least 95% sequence identity to SEQ ID NO: 3, wherein the polymerase comprises a mutation, relative to SEQ ID NO: 3, at an amino acid position selected from the group consisting of 526-534, 551-560, 570-590, 609-630, 655-663, 705-720, 728-730, and 770-829, wherein the amino acid position numbering is according to SEQ ID NO: 2.

Embodiment 14. The polymerase of embodiment 13, wherein the mutation is at amino acid position 360, 531, 538, 558, 566, 567, 568, 573, 579, 580, 583, 622, 628, 629, 630, 635, 636, 637, 640, 716, 718, 782, 785, 786, 806, 815, 819, 826, or 827, wherein the amino acid position numbering is according to SEQ ID NO: 2.

Embodiment 15. The polymerase of embodiment 13, wherein the mutation is L360P, P531Q, L538F, A558M, P566L, Y567H, H568P, N573E, Q579N, L580D, L580G, L583N, L583V, N622G, I628L, I628M, R629A, L630D, L630P, K635S, I636M, I636L, R637M, F640V, I716Q, D718K, N782R, S785R, F786R, K806E, K806M, L815F, R819K, R819M, R819P, R819V, L826Y or Q827D, wherein the amino acid position numbering is according to SEQ ID NO: 2.

Embodiment 16. The polymerase of embodiment 13, wherein the polymerase comprises at least two mutations, relative to SEQ ID NO: 1, at amino acid positions selected from the group consisting of 526-534, 551-560, 570-590, 609-630, 655-663, 705-720, 728-730, and 770-829, wherein the amino acid position numbering is according to SEQ ID NO: 2.

Embodiment 17. The polymerase of embodiment 16, wherein the at least two mutations are at amino acid positions 360, 531, 538, 558, 566, 567, 568, 573, 579, 580, 583, 622, 628, 629, 630, 635, 636, 637, 640, 716, 718, 782, 785, 786, 806, 815, 819, 826, or 827, wherein the amino acid position numbering is according to SEQ ID NO: 2.

Embodiment 18. The polymerase of embodiment 16, wherein the at least two mutations comprise two or more of L360P, P531Q, L538F, A558M, P566L, Y567H, H568P, N573E, Q579N, L580D, L580G, L583N, L583V, N622G, I628L, I628M, R629A, L630D, L630P, K635S, I636M, I636L, R637M, F640V, I716Q, D718K, N782R, S785R, F786R, K806E, K806M, L815F, R819K, R819M, R819P, R819V, L826Y or Q827D, wherein the amino acid position numbering is according to SEQ ID NO: 2.

Embodiment 19. The polymerase of any one of embodiments 1-18, wherein the polymerase comprises a mutation at amino acid position 628, wherein the amino acid position numbering is according to SEQ ID NO: 2.

Embodiment 20. The polymerase of embodiment 19, wherein the polymerase comprises a I628L mutation, wherein the amino acid position numbering is according to SEQ ID NO: 2.

Embodiment 21. The polymerase of embodiment 20, wherein the polymerase comprises a 785R mutation, wherein the amino acid position numbering is according to SEQ ID NO: 2.

Embodiment 22. The polymerase of any one of embodiments 1-18, wherein the polymerase comprises a mutation at amino acid position 630, wherein the amino acid position numbering is according to SEQ ID NO: 2.

Embodiment 23. The polymerase of embodiment 22, wherein the polymerase comprises a L630D mutation, wherein the amino acid position numbering is according to SEQ ID NO: 2.

Embodiment 24. The polymerase of embodiment 22, wherein the polymerase comprises a L630P mutation, wherein the amino acid position numbering is according to SEQ ID NO: 2.

Embodiment 25. The polymerase of any one of embodiments 1-18, wherein the polymerase comprises a mutation at amino acid position 785, wherein the amino acid position numbering is according to SEQ ID NO: 2.

Embodiment 26. The polymerase of embodiment 25, wherein the polymerase comprises an S785R mutation, wherein the amino acid position numbering is according to SEQ ID NO: 2.

Embodiment 27. The polymerase of any one of embodiments 1-18, wherein the polymerase comprises a mutation at amino acid position 827, wherein the amino acid position numbering is according to SEQ ID NO: 2.

Embodiment 28. The polymerase of embodiment 27, wherein the polymerase comprises a Q827D mutation, wherein the amino acid position numbering is according to SEQ ID NO: 2.

Embodiment 29. A composition comprising the polymerase of any one of embodiments 1-28, and an aqueous solution.

Embodiment 30. The composition of embodiment 29, further comprising nucleotides and a nucleic acid hybrid comprising a target nucleic acid molecule hybridized to a sequencing primer.

Embodiment 31. The composition of embodiment 30, wherein at least a portion of the nucleotides are labeled nucleotides.

Embodiment 32. The composition of embodiment 31, wherein the labeled nucleotides comprise a fluorescent label.

Embodiment 33. A nucleic acid molecule encoding the polymerase of any one of embodiments 1-28.

Embodiment 34. An expression vector comprising the nucleic acid molecule of embodiment 33.

Embodiment 35. A host cell, comprising the expression vector of embodiment 34.

Embodiment 36. A method of making a polymerase, comprising:
culturing the host cell of embodiment 35;
expressing, using the host cell, the polymerase; and
isolating the polymerase.

Embodiment 37. A method, comprising: providing in a reaction mixture (i) a nucleic acid molecule, (ii) a labeled nucleotide, and (iii) the mutant polymerase of any one of embodiments 1-28, under conditions sufficient to extend the nucleic acid molecule with the labeled nucleotide.

Embodiment 38. A method, comprising: contacting a labeled nucleotide with the mutant polymerase of any one of embodiments 1-28 and extending a nucleic acid molecule to incorporate the labeled nucleotide.

Embodiment 39. The method of embodiments 37 or 38, wherein said labeled nucleotide comprises a fluorescent dye.

Embodiment 40. A method, comprising:
(a) forming a reaction mixture comprising:
   (i) a template oligonucleotide sequence comprising a primer binding site, one or more nucleotide residues comprising the same base, and a first member of a fluorescence probe pair;
   (ii) a primer sequence configured to hybridize to the primer binding site;
   (iii) a first nucleotide set where a member of the first nucleotide set comprises a second member of the fluorescence probe pair, and where the first nucleotide set comprises one or more nucleotides that are not complementary to the one or more nucleotide residues;
   (iv) a second nucleotide set comprising one or more nucleotides that are complementary to the one or more nucleotide residues; and
   (v) a polymerase; and
(b) detecting a presence or absence of a fluorescence resonance energy transfer (FRET) signal, where a change in the FRET signal indicates that the polymerase has incorporated nucleotides of the first nucleotide set or has incorporated nucleotides of the second nucleotide set to extend the primer sequence through the one or more nucleotide residues.

### EXAMPLES

### Example 1—Comparison of Nucleotide Incorporation Rates for Mutant Polymerases Using a FRET-Based Polymerase Assay

The experimental procedure for performing the FRET-based multiple nucleotide incorporation assay described herein was as follows. The template oligonucleotide used in the assay was dissolved to a final concentration of 10 nM in a buffer of 20 mM Tris-HCl, pH 8.8, 2 mM MgCl₂, 110 mM NaCl. The two labeled complimentary nucleotides were then added to this solution, one labeled with Atto532 (the fluorescence donor) and the other labeled with Atto633 (the fluorescence acceptor), to a final concentration of 100 nM. Aliquots of this solution were then pipetted into individual wells of a 96 well plate and the plate was paced in a fluorescence microplate reader, pre-equilibrated to a temperature of 45° C. The fluorescence detection settings were set at two excitation/emission wavelength combinations, 490 nm/560 nm (to monitor Atto532 emission) and 490 nm/660 nm (to monitor Atto532—Atto633 FRET-based emission). The fluorescence signals were monitored over the next 5-6 minutes and, once a stable baseline was achieved, the reading was interrupted to add aliquots of the polymerases being tested, typically to a final concentration of 20-40 nM. The fluorescence signals were then monitored for another 45-60 minutes. Typically, control wells contain either no polymerase, or only donor-labeled, or only acceptor-labeled nucleotides.

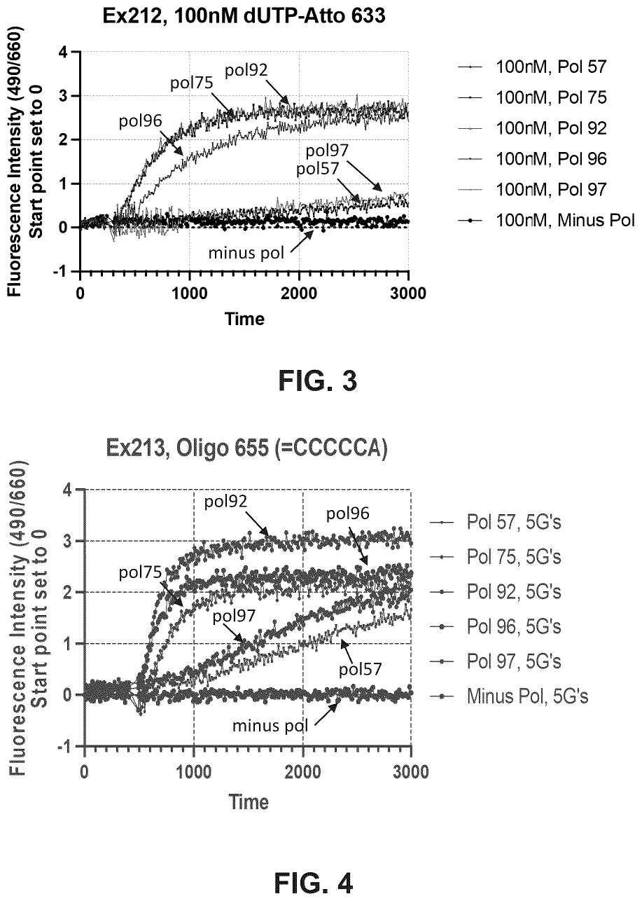

FIG. 3 provides a non-limiting example of results from an assay where five different polymerase mutants were tested with an oligonucleotide having the template sequence . . . TTTA and the labeled complementary nucleotides. Fluorescence traces were measured as a function of time (seconds) using 490 nm excitation light while monitoring the Atto 532—Atto633 FRET-based emission at 660 nm. As can be seen, Pol75 and Pol92 show the fastest nucleotide incorporation rates, followed by Pol96, Pol57, and Pol97 as the slowest.

FIG. 4 provides another example using the template sequence . . . CCCCCA. The donor-labeled and acceptor-labeled nucleotides were dGTP-Atto532 and dUTP-Atto632, respectively.

Here too, the different incorporation kinetics of the polymerase mutants tested are clearly distinguishable by using 490 nm excitation light while monitoring the Atto 532—Atto633 FRET-based emission at 660 nm.

### Example 2—FRET-Based Polymerase Assay of Nucleotide Incorporation Rates for Mutant Polymerases Using Three Fluorophores

The experimental procedure for performing the FRET-based multiple nucleotide incorporation assay using three fluorophores as described herein was as follows. The template oligonucleotide (comprising a Pacific Blue fluorophore—the first fluorescence donor—attached to the 5′ end) was dissolved to a final concentration of 10 nM in a buffer of 20 mM Tris-HCl, pH 8.8, 2 mM MgCl₂, 110 mM NaCl. The two labeled complementary nucleotides were then added to this solution, one labeled with Atto532 (the first fluorescence acceptor/second fluorescence donor) and the other labeled with Atto633 (the second fluorescence acceptor), to a final concentration of 100-200 nM. Aliquots of this solution were then pipetted into individual wells of a 96 well plate and the plate was paced in a fluorescence microplate reader, pre-equilibrated to a temperature of 45° C. The fluorescence detection settings were set at an excitation/emission wavelength combination of 406 nm/460 nm to monitor Pacific Blue fluorescence, at 406 nm/560 nm to monitor Pacific Blue—Atto532 FRET-based emission, and at 520 nm/660 nm to monitor Pacific blue—Atto532—Atto633 FRET-based emission. The fluorescence signals were monitored over the next 5-6 minutes and, once a stable baseline was achieved, the reading was interrupted to add aliquots of the polymerases being tested, typically to a final concentration of 20-40 nM. The fluorescence signals were then monitored for another 45-60 minutes. Typically, control wells contain either no polymerase, or only donor-labeled or only acceptor-labeled nucleotides.

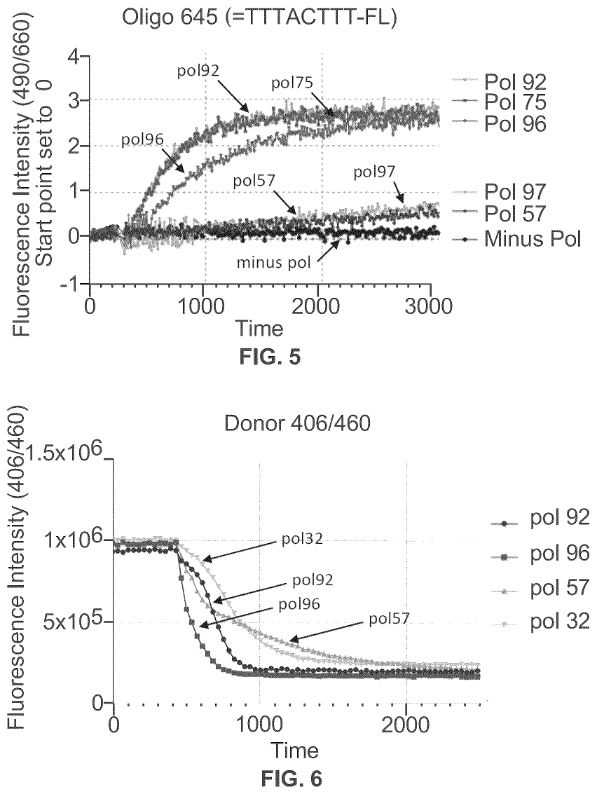

FIG. 5 provides a non-limiting example of results from an assay where five different polymerase mutants were tested with an oligonucleotide having the template sequence . . . TTTACTTT-Fluorescein. Although the oligonucleotide in this examples was fluorescently labeled, it was not used to generate FRET signals. Rather, the two labeled complementary nucleotides, dATP-linker-Atto 532 and dUTP-linker-Atto633, were used in the same assay format as described for Example 1 (i.e., Atto532 was excited at 490 nm and Atto532—Atto633 FRET-based emission was monitored at 660 nm).

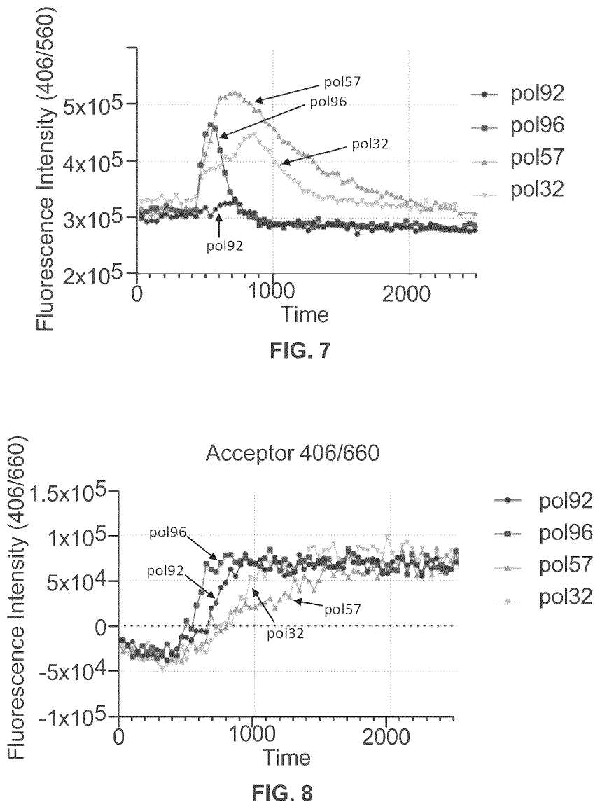

FIG. 6 provides a non-limiting example of results for an assay where four different polymerase mutants were tested using an oligonucleotide having the template sequence . . . TTTGCTTT-Pacific Blue. In this experiment, Pacific Blue was used as the first fluorescence donor while the two labeled complementary nucleotides, dATP-linker-Atto532 (first fluorescence acceptor/second fluorescence donor) and dCTP-linker-Atto633 (second fluorescence acceptor), were used to generate a FRET-based signal monitored at 660 nm. FIG. 6 illustrates the fluorescence signals recorded as a function of time for Pacific Blue fluorescence (406 nm excitation/460 nm emission) following addition of the polymerase. The signal decreases in intensity as the Atto532-labeled nucleotides are incorporated by the polymerase, thereby providing an energy acceptor for the Pacific Blue label. FIG. 7 provides a non-limiting example of the “intermediate” fluorescence traces, i.e., the fluorescence signals recorded as a function of time for the Pacific Blue—Atto532 FRET-based signal (406 nm excitation/560 nm emission). There is a transient increase in signal as the polymerase incorporates more of the Atto532-labeled complementary nucleotide, but the signal decreases again as the Atto633-labeled complementary nucleotide is incorporated into the template oligonucleotide sequence. FIG. 8 provides a non-limiting example of the Pacific Blue—Atto532—Atto633 FRET-based signal (406 nm excitation/660 nm emission). The signal rises and then plateaus as the primer extension reaction reaches completion. Note the fluorescence traces for the Pol92 and Pol96 polymerases. Using the original non-modified assay, These two polymerases appear to function similarly using the two fluorophore (single FRET probe pair) assay format described in Example 1, with Pol92 perhaps functioning slightly better. However, when these polymerases were tested for use in a sequencing reaction, it was discovered that Pol96 unexpectedly outperformed Pol92. Pol96 is apparently more efficient than Pol92 at incorporating labeled nucleotides. This can be observed in the “intermediate” fluorescence kinetic traces shown in FIG. 7. Pol96 exhibited a sharper peak for each tested oligo, indicating that it more quickly incorporates the first and second labeled nucleotides.

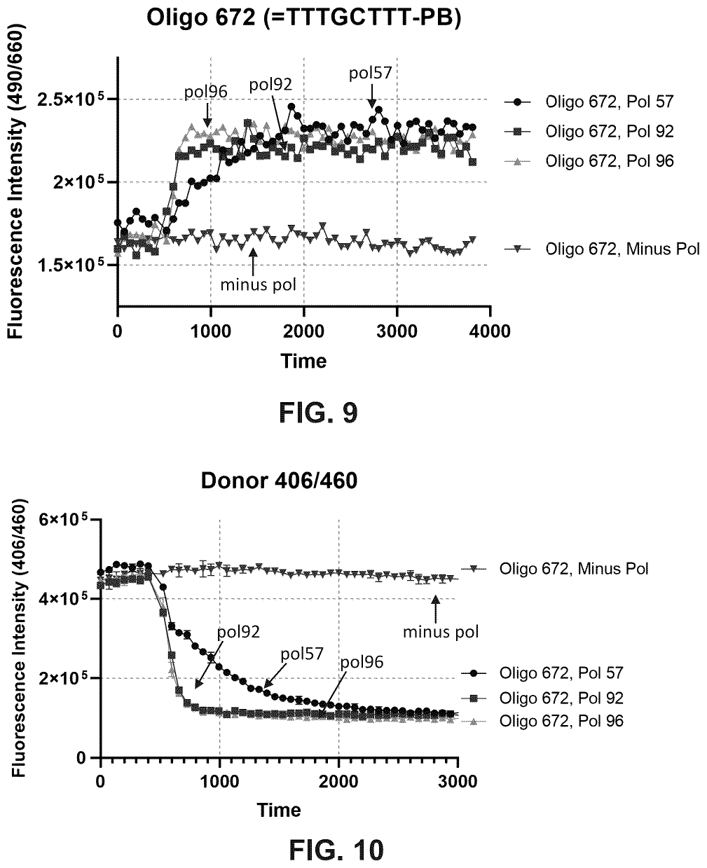

FIG. 9 provides another example of data for the two fluorophore (single FRET probe pair) assay format. In this case, three mutant polymerases were tested using an oligonucleotide having the template sequence . . . TTTGCTTT-Pacific Blue. Although the oligonucleotide in this examples was fluorescently labeled, it was not used to generate FRET signals. Rather, the two labeled complementary nucleotides, dATP-linker-Atto532 and dUTP-linker-Atto633, were used in the same assay format as described for Example 1 (i.e., Atto532 was excited at 490 nm and Atto532—Atto633 FRET-based emission was monitored at 660 nm).

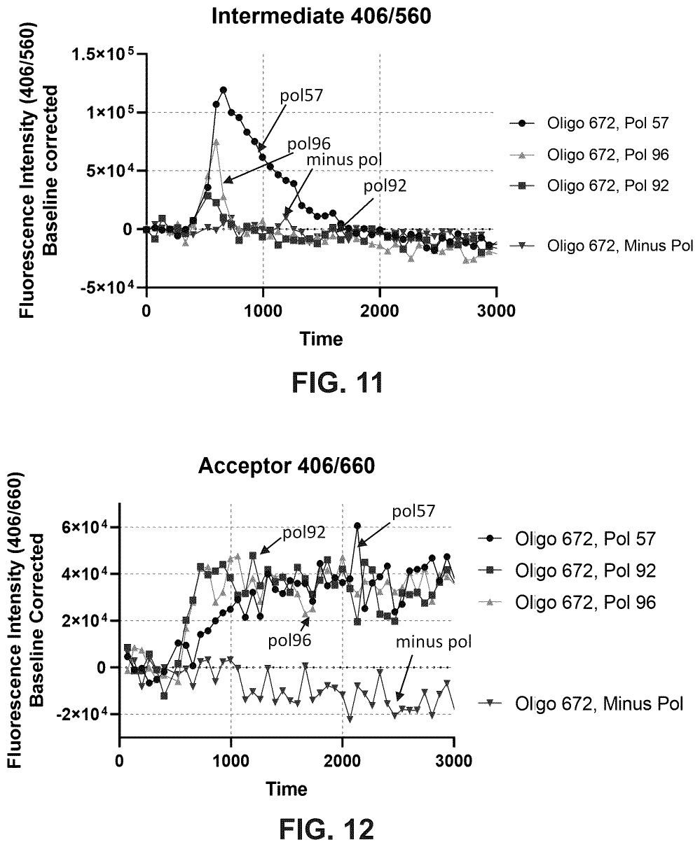

FIG. 10 provides a non-limiting example of results for an assay where three different polymerase mutants were tested using an oligonucleotide having the template sequence . . . TTTGCTTT-Pacific Blue. In this experiment, Pacific Blue was again used as the first fluorescence donor while the two labeled complementary nucleotides, dATP-linker-Atto532 (first fluorescence acceptor/second fluorescence donor) and dCTP-linker-Atto633 (second fluorescence acceptor), were used to generate a FRET-based signal monitored at 660 nm. FIG. 10 illustrates the fluorescence signals recorded as a function of time for Pacific Blue fluorescence (406 nm excitation/460 nm emission) following addition of the polymerase. FIG. 11 provides a non-limiting example of the “intermediate” fluorescence traces, i.e., the fluorescence signals recorded as a function of time for the Pacific Blue—Atto532 FRET-based signal (406 nm excitation/560 nm emission). FIG. 12 provides a non-limiting example of the Pacific Blue—Atto532—Atto633 FRET-based signal (406 nm excitation/660 nm emission). The fluorescence signal traces in all three figures—FIGS. 10-12—show similar kinetics as those illustrated in FIGS. 6-8.

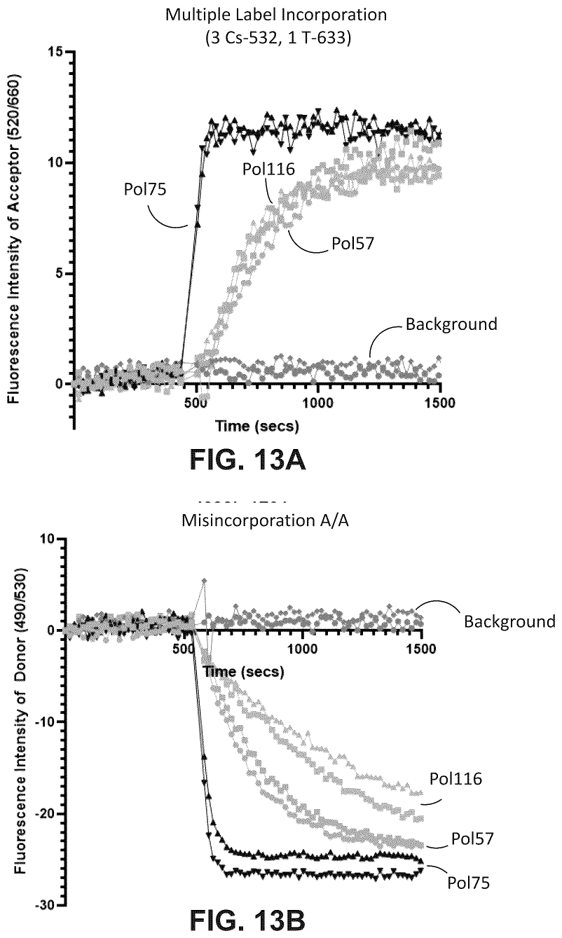

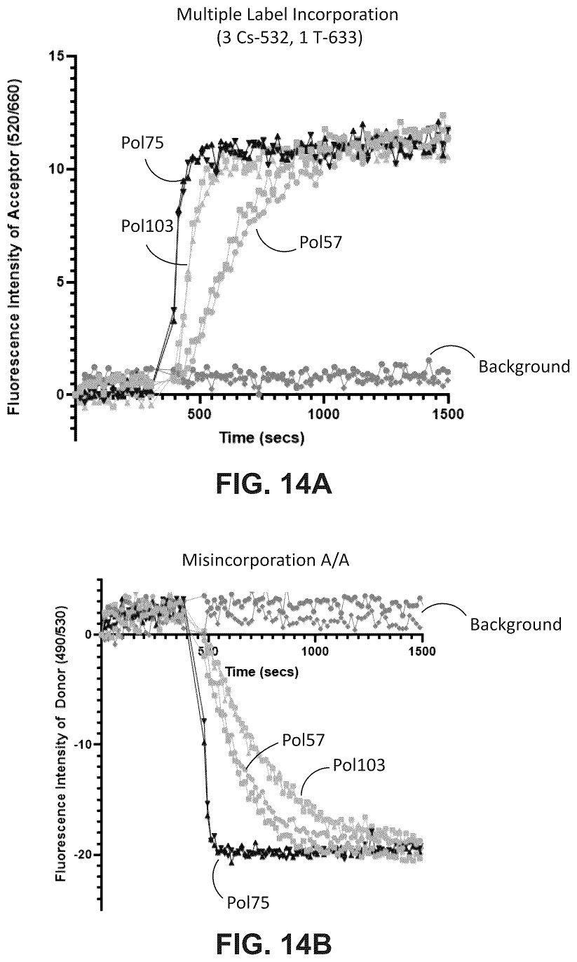

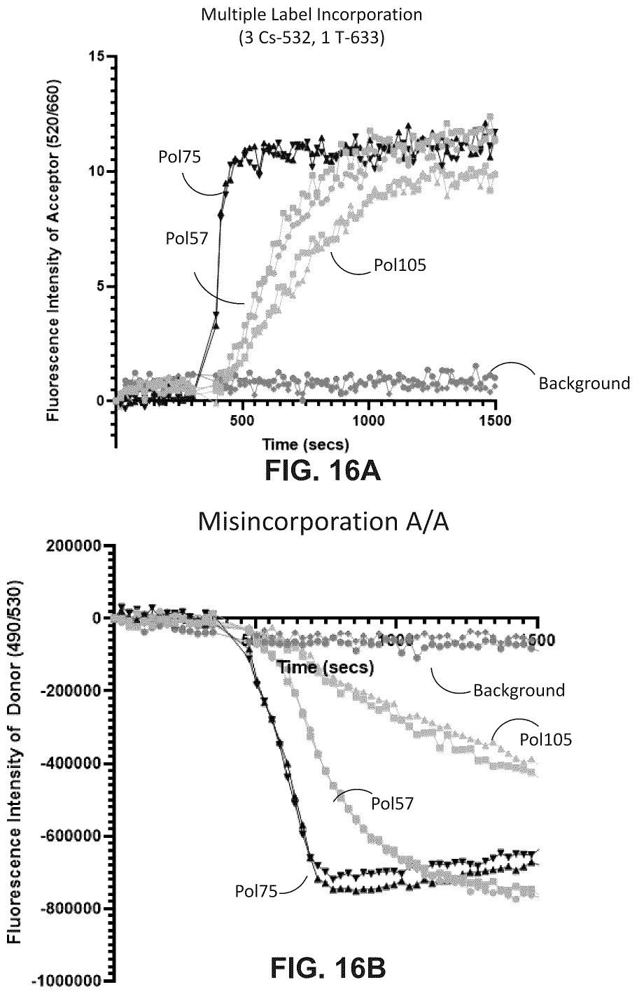

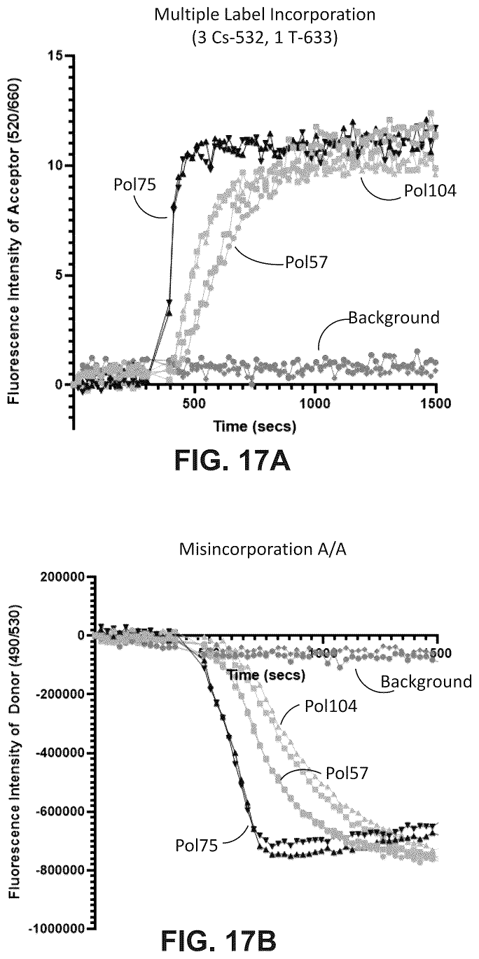

FIGS. 13A, 14A, 15A, 16A, and 17A provide non-limiting examples of results from assays where five different polymerases were tested with an oligonucleotide having the template sequence . . . GACGGGACTTT and two labeled complementary nucleotides, dCTP-Atto532 (fluorescence donor) and dUTP-Atto633 (fluorescence acceptor). Fluorescence traces were measured as a function of time (seconds) using 530 nm excitation light while monitoring Atto633 emission at 660 nm. Pol75 consistently demonstrates the highest rate of incorporation. Pol103 demonstrates an incorporation rate that is most similar to that of Pol75 among this set of five polymerase mutants (see FIG. 14A).

### Example 3—FRET-Based Polymerase Assay of Nucleotide Misincorporation Rates for Mutant Polymerases

The experimental procedure for the FRET-based nucleotide misincorporation assay using three fluorophores as described herein was as follows. The template oligonucleotide (comprising a fluorescein fluorophore—the fluorescence donor—attached to the 5′ end) was dissolved to a final concentration of 10 nM in a buffer of 20 mM Tris-HCl, pH 8.8, 2 mM MgCl₂, 110 mM NaCl. The labeled non-complementary nucleotides were added prior to, subsequent, or simultaneously with the unlabeled complementary nucleotides. The labeled non-complementary nucleotides were labeled with Atto633 (the fluorescence acceptor) attached to the 5′ end, to a final concentration of 100-200 nM. In some instances, the final concentration of the labeled non-complementary nucleotides was 200-500 nM. In some instances, the final concentration of the labeled non-complementary nucleotides was 500 nM-1 μM. Aliquots of this solution were then pipetted into individual wells of a 96 well plate and the plate was paced in a fluorescence microplate reader, pre-equilibrated to a temperature of 45° C. The fluorescence detection settings were set at an excitation/emission wavelength combination of 490/660 to monitor fluorescein—Atto633 FRET-based emission. The fluorescence detection settings were set at an excitation/emission wavelength combination of 490 nm/530 nm to monitor fluorescein fluorescence. The fluorescence signals were monitored over the next 5-6 minutes and, once a stable baseline was achieved, the reading was interrupted to add aliquots of the polymerases being tested, typically to a final concentration of 20-50 nM. The fluorescence signals were then monitored for another 20-60 minutes. Typically, control wells contain either no polymerase, or only unlabeled or only acceptor-labeled nucleotides.

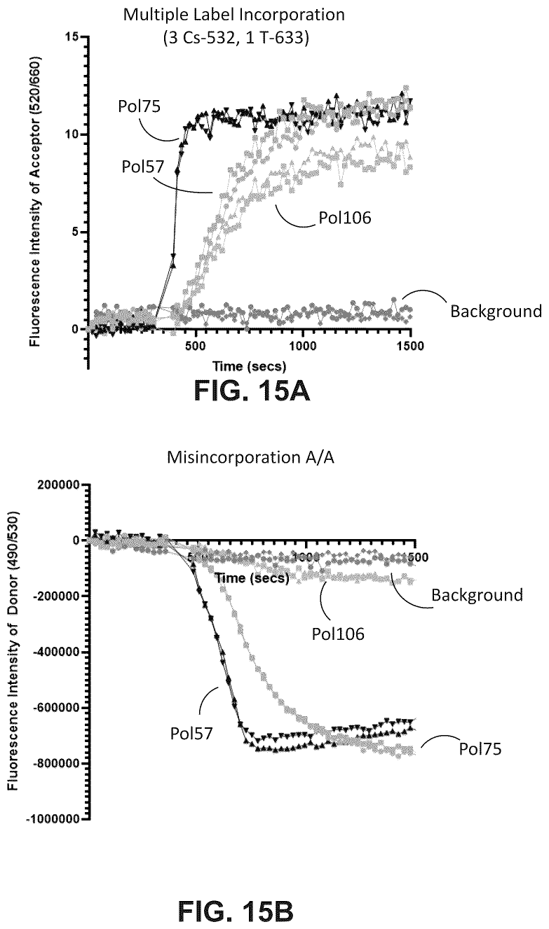

FIGS. 13B, 14B, 15B, 16B, and 17B provide non-limiting examples of results from assays where five different polymerase mutants (e.g., corresponding to the polymerase mutants in FIGS. 13A, 14A, 15A, 16A, and 17A) were tested with an oligonucleotide having the template sequence . . . TCAGACCAGCTATTT-Fluorescein, labeled non-complementary nucleotides (e.g., dATP-Atto633), and non-labeled complementary nucleotides (e.g., dTTPs). Fluorescence traces were measured as a function of time (seconds) using 490 nm excitation light while monitoring fluorescein emission at 530 nm and/or Atto633 emission at 660 nm. Pol57 and Pol75 serve as controls for these assays. As can be seen, Pol75 consistently displays the fastest misincorporation. Polymerases that display shallower misincorporation fluorescence curves are those that have a lower predilection for incorporating non-Watson-Crick base pairs (e.g., decreased rate of misincorporation). Each of the five different mutant polymerases assayed here displayed a decreased rate of misincorporation compared with both Pol57 and Pol75, with Pol106 displaying the lowest rate of misincorporation (see FIG. 15B).

### Example 4—Hybrid BST Polymerase Mutant Library

Approximately 150 amino acid residues in the BST polymerase were selected for mutagenesis study, as shown in FIG. 20. For simplicity, SEQ ID NO.:1 is used to show where the amino acids located within the polymerase fragment.

The hybrid protein in SEQ ID NO.: 3 was used to construct the polymerase mutant library. The resulting library contains polymerase mutants that contain one or more mutations at amino acid positions 495, 515, 526-534, 538, 549, 551-568, 570-590, 609-630, 635-637, 640, 655-663, 674, 681, 683, 688, 689, 701, 703-720, 728-730, 743, 761, 770-829, 872, and 873, with amino acid numbering according to SEQ ID NO: 2. For consistency, numbering of the mutant location follows the numbering of the polymerase domain, as illustrated in FIG. 20.

### Example 5—Activities of Polymerase Mutants

The comparison of experimental results (e.g., assays such as described in for different polymerases can be used to identify individual mutations, or combinations of mutations, that have desired effects on enzyme performance. Qualitative results from both misincorporation assays (e.g., as described in Example 3) and incorporation assays (e.g., as described in Examples 1 and 2) are provided. Both misincorporation and incorporation rates for mutant polymerases were normalized to the misincorporation and incorporation rates for Pol57. Table 5 indicates the key for the qualitative values in Table 4. Polymerases with decreased misincorporation (e.g., <1 normalized values) and increased incorporation rates (e.g., >1 normalized values) were selected for additional analysis.

TABLE 4
Non-limiting polymerase compositions.

| | | | Qualitative | Qualitative | Qualitative | Qualitative | Qualitative |
|---|---|---|---|---|---|---|---|
| | | | A opp A | multi-C | multi-G | G opp T | T opp G |
| Mutation | Polymerase | | misincorporation | incorporation | incorporation | misincorporation | misincorporation |
| position(s)** | # | SEQ ID NO | rate | rate | rate | rate | rate |
| D718K | 18 | | −− | −− | − | −− | −− |
| D718K + N782R | 19 | | −− | − | n.d | n.d. | n.d. |
| F786R | 32 | | + | + | n.d | n.d | n.d. |
| n/a | 57 | SEQ ID NO.: 3 | 1 | 1 | 1 | 1 | 1 |
| K549I + A674V | 61 | | −− | −− | −− | −− | −− |
| V661I + L662M + | 75 | | +++ | ++ | +++ | ++ | +++ |
| I68IV + T683K + | | | | | | | |
| M701L + F743Y + | | | | | | | |
| A800G + I689V + | | | | | | | |
| Q704I + V713M | | | | | | | |
| Q827D | 103 | SEQ ID NO.: 10 | − | ++ | +++ | + | n.d. |
| K635S | 104 | | − | + | +++ | − | n.d. |
| R629A | 105 | | −− | − | −−− | − | n.d. |
| R637M + L630P | 106 | | −−− | − | −−− | − | n.d. |
| F640V | 109 | | − | − | +++ | − | n.d. |
| I636M | 111 | | − | − | +++ | − | n.d. |
| I636L | 112 | | − | − | +++ | − | n.d. |
| R819V | 113 | | − | − | +++ | + | n.d. |
| R819P | 114 | | − | − | +++ | − | n.d |
| R819M | 115 | | − | − | +++ | + | n.d. |
| I628L | 116 | SEQ ID NO.: 7 | −− | + | − | − | −− |
| I716Q | 117 | | − | + | +++ | + | n.d. |
| I628M | 119 | | − | + | +++ | + | n.d. |
| R819K | 120 | | − | + | +++ | + | n.d. |
| L826Y | 121 | | −− | −− | +++ | − | n.d. |
| K806E | 122 | | − | + | +++ | + | n.d. |
| K806M | 123 | | − | 1 | +++ | + | n.d. |
| V661I + L662M + | 127 | | + | n.d. | + | n.d | n.d. |
| I681V + T683K + | | | | | | | |
| M701L + F710Y + | | | | | | | |
| A800G + I689V | | | | | | | |
| N573E | 128 | | − | + | +++ | − | n.d. |
| N622G | 129 | | −− | − | +++ | + | n.d. |
| L583N | 130 | | − | − | −− | − | − |
| A558M | 131 | | −− | − | +++ | − | n.d. |
| L580D | 132 | | −− | − | +++ | − | n.d. |
| P531Q | 133 | | − | + | +++ | − | n.d. |
| L538F | 134 | | − | + | +++ | − | n.d. |
| Q579N | 135 | | −− | − | +++ | − | n.d. |
| L815F | 136 | | − | − | +++ | − | n.d. |
| S785R + I716Q | 138 | | − | + | +++ | + | n.d. |
| H568P | 142 | | −− | −− | n.d. | − | − |
| L583V | 143 | | − | + | n.d. | + | − |
| Y567H | 144 | | + | + | n.d | + | + |
| P566L | 145 | | + | + | n.d. | + | + |
| L580G | 146 | | − | + | n.d. | + | − |
| SS04 S785R + I628L | 147 | SEQ ID NO.: 6 | −− | + | − | − | −− |
| I628L + L583N | 149 | | −− | − | −− | −− | −− |
| L630D | 151 | SEQ ID NO.: 8 | −− | + | + | + | − |
| L630P | 152 | SEQ ID NO.: 9 | −− | + | + | − | −− |
| I628L + D718K + | 154 | | −−− | − | −− | −− | −−− |
| N782R | | | | | | | |
| I628L + Q827D | 156 | | − | + | + | + | −− |
| V661I + L662M + | 157 | | ++ | ++ | ++ | ++ | +++ |
| I68IV + T683K + | | | | | | | |
| M701L + F743Y + | | | | | | | |
| A800G + I689V + | | | | | | | |
| Q704I + V713M + | | | | | | | |
| L583N | | | | | | | |
| I628L + L630P | 158 | | −− | ++ | + | − | −− |
| I628L + Q579N | 159 | | −− | − | −− | + | −− |
| A707Y | 160 | | −− | − | − | −− | −− |
| L575I | 161 | | −− | − | − | − | − |
| E572D | 162 | | − | − | − | + | −− |
| A565R | 163 | | − | + | − | + | − |
| E623Y | 164 | | −− | −− | + | + | − |
| N782V | 166 | | + | + | + | + | + |
| V661I + L662M + | 167 | | ++ | +++ | +++ | ++ | +++ |
| I68IV + T683K + | | | | | | | |
| M701L + F743Y + | | | | | | | |
| A800G + I689V + | | | | | | | |
| Q704I + V713M + | | | | | | | |
| L580D | | | | | | | |
| I628L + L583V | 168 | | −− | + | + | − | −− |
| L561I | 169 | | − | − | + | + | + |
| E562N | 170 | | − | + | + | ++ | + |
| K563G | 171 | | − | − | + | + | + |
| L564V | 172 | | + | + | + | + | + |
| I628L + A707Y | 175 | | −− | + | − | −− | −−− |
| I628L + L630D | 178 | | −− | + | + | − | −− |
| V661I + L662M + | 179 | | ++ | ++ | ++ | ++ | +++ |
| I68IV + T683K + | | | | | | | |
| M701L + F743Y + | | | | | | | |
| A800G + I689V + | | | | | | | |
| Q704I + V713M + | | | | | | | |
| L580D + L583V | | | | | | | |
| I628L + Q579N + | 180 | | −−− | −− | −− | −− | −−− |
| D718K | | | | | | | |
| I628L + L583V + | 181 | | −− | − | + | −− | −−− |
| D718K | | | | | | | |
| I628L + L583V + | 182 | | −− | + | ++ | − | −− |
| L630D | | | | | | | |
| V661I + L662M + | 183 | | ++ | ++ | ++ | ++ | +++ |
| I68IV + T683K + | | | | | | | |
| M701L + F743Y + | | | | | | | |
| A800G + I689V + | | | | | | | |
| Q704I + V713M + | | | | | | | |
| L580D + L583V + | | | | | | | |
| S785R | | | | | | | |
| S655N | 186 | | + | + | + | ++ | + |
| R703A | 187 | | −− | ++ | + | −− | −− |
| Q827D + S785R | 188 | | + | ++ | + | + | − |
| R637M + I628M | 189 | | −−− | −−− | − | −− | −−− |
| I628M + D718A | 190 | | −− | + | + | + | − |
| R637M + L630P + | 191 | | −−− | − | − | −− | −−− |
| D718K | | | | | | | |
| I628L + Q579N + | 192 | | −− | + | + | − | − |
| F786R | | | | | | | |
| R637M + I628M + | 193 | | −−− | −− | n.d. | − | −− |
| D718A | | | | | | | |
| I628M + D718K | 194 | | −− | − | − | −− | −− |
| R703H | 195 | | −− | − | −− | −−− | −−− |
| R637M + I628M + | 196 | | −− | −−− | − | −− | |
| D718K | | | | | | | |
| R703P | 197 | | −− | + | + | −− | −− |
| G761E | 198 | | − | + | + | − | + |
| V495F | 199 | | − | + | + | − | −− |
| W872H | 200 | | − | + | + | − | + |
| F743R | 201 | | − | − | 1 | − | 1 |
| I628M + R703A | 205 | | −− | ++ | + | −− | −− |
| R703A + D718K | 206 | | −− | + | −− | −−− | −− |
| I628M + R703A + | 207 | | −− | ++ | + | −−− | −−− |
| D718K | | | | | | | |
| R660C | 208 | | − | + | 1 | − | − |
| D688M | 209 | | − | − | 1 | − | − |
| I689Q | 210 | | + | + | 1 | − | − |
| V661I + L662M + | 211 | | ++ | +++ | n.d. | ++ | +++ |
| I68IV + T683K + | | | | | | | |
| M701L + F743Y + | | | | | | | |
| A800G + I689V + | | | | | | | |
| Q704I + V713M + | | | | | | | |
| D718A | | | | | | | |
| V661I + L662M + | 212 | | + | ++ | n.d. | ++ | +++ |
| I68IV + T683K + | | | | | | | |
| M701L + F743Y + | | | | | | | |
| A800G + I689V + | | | | | | | |
| Q704I + + F710Y + | | | | | | | |
| L580D + L583V + | | | | | | | |
| S785R | | | | | | | |
| V661I + L662M + | 213 | | ++ | +++ | n.d. | + | +++ |
| I68IV + T683K + | | | | | | | |
| M701L + A800G + | | | | | | | |
| I689V + Q704I + | | | | | | | |
| V713M | | | | | | | |
| Y873D | 214 | | −− | + | n.d | − | − |
| W872H + D718K | 215 | | −− | − | n.d. | − | −− |
| M701L | 217 | | − | − | − | − | − |
| M701L + Q704I | 218 | | −− | −− | − | −− | − |
| V661I + L662M | 219 | | − | −− | − | − | − |
| I689V + T683K | 220 | | ++ | ++ | ++ | ++ | ++ |
| F743Y | 221 | | + | + | + | + | + |
| V713M | 222 | | − | − | 1 | −− | −− |
| I689V | 223 | | + | + | + | + | 1 |
| V661I + L662M + | 224 | | n.d. | n.d. | ++ | n.d. | n.d. |
| I68IV + T683K + | | | | | | | |
| M701L + F743Y + | | | | | | | |
| A800G + I689V + | | | | | | | |
| Q704I + V713M + | | | | | | | |
| D718K + I628M | | | | | | | |
| V661I + L662M + | 225 | | n.d. | n.d. | ++ | n.d. | n.d. |
| I68IV + T683K + | | | | | | | |
| M701L + F743Y + | | | | | | | |
| A800G + I689V + | | | | | | | |
| Q704I + V713M + | | | | | | | |
| I628M | | | | | | | |
| V661I + L662M + | 226 | | n.d. | n.d. | +++ | n.d. | n.d. |
| I68IV + T683K + | | | | | | | |
| M701L + F743Y + | | | | | | | |
| A800G + I689V + | | | | | | | |
| Q704I + V713M + | | | | | | | |
| A707Y | | | | | | | |
| N782R | 227 | | + | − | + | + | − |
| V661I + L662M + | 228 | | n.d. | n.d. | +++ | n.d. | n.d. |
| I68IV + T683K + | | | | | | | |
| M701L + F743Y + | | | | | | | |
| A800G + I689V + | | | | | | | |
| Q704I + V713M + | | | | | | | |
| L630P | | | | | | | |
| D559K | 229 | | − | − | − | + | − |
| D559S | 230 | | + | − | 1 | + | − |
| E788R | 231 | | + | − | 1 | + | + |
| N780K | 232 | | − | − | + | + | 1 |
| N529R | 233 | | − | − | + | + | − |
| D559R | 234 | | − | + | + | + | − |
| E515K | 235 | | | + | + | + | 1 |
| V661I + L662I + | 236 | | n.d. | n.d. | +++ | n.d. | n.d. |
| I68IV + T683K + | | | | | | | |
| M701L + F743Y + | | | | | | | |
| A800G + I689V + | | | | | | | |
| Q704I + V713M | | | | | | | |
| V661I + L662M + | 237 | | n.d. | n.d. | ++ | n.d. | n.d. |
| I68IV + T683F + | | | | | | | |
| M701L + F743Y + | | | | | | | |
| A800G + I689V + | | | | | | | |
| Q704I + V713M | | | | | | | |
| V661I + L662M + | 238 | | n.d. | n.d. | +++ | n.d. | n.d. |
| I68IV + T683K + | | | | | | | |
| M701L + F743Y + | | | | | | | |
| A800G + I689Q + | | | | | | | |
| Q704I + V713M | | | | | | | |
| I689F | 243 | | ++ | ++ | ++ | ++ | ++ |
| T683K | 244 | | ++ | ++ | ++ | ++ | ++ |
| T683V | 245 | | + | + | ++ | + | + |
| I689Y | 246 | | ++ | ++ | +++ | ++ | ++ |

n/a—not applicable
n.d.—not determined
**All mutation positions are identified based on the numbering of the full-length wildtype Bst polymerase in SEQ ID NO.: 2.

TABLE 5
Key to normalized values for qualitative comparisons in Table 4.

| Normalized mis- | Qualitative | Normalized multi- | Qualitative |
|---|---|---|---|
| incorporation value | mis- | incorporation value | multiple |
| (e.g., compared to | incorporation | (e.g., compared to | incorporation |
| Pol57 value) | indication | Pol57 value) | indication |
| >4 | +++ | >6 | +++ |
| >2-4 | ++ | >3-6 | ++ |
| >1-2 | + | >1-3 | + |
| 1 | 1 | 1 | 1 |
| <1 | − | <1 | − |
| <0.5 | −− | <0.5 | −− |
| <0.1 | −−− | <0.1 | −−− |

As can be seen in Table 4, different mutations at an individual amino acid positions can have similar effects on enzyme performance. For example, polymerases 151 and 152, have similar qualitative performance with regards to misincorporation and multiple nucleotide incorporation, and both of these polymerases comprise amino acid mutations at the same position, L630D and L630P, respectively. Here, an amino acid with a hydrophobic side chain (i.e., leucine, L) has been replaced with either an amino acid with a negatively charged side chain (i.e., aspartic acid, D) or an amino acid with a bulky side chain (e.g., proline, P), a surprising result. An in silico study would not necessarily have predicted that such amino acid substitutions would have similar effects on enzyme activity. Thus, enzymatic screens in vitro can be an essential component of identifying novel mutant enzymes with desired properties.

### Example 6: Polymerase Mutants Sequencing Results

Sequencing assays were performed for certain mutant polymerases. Mutant polymerases were selected for sequencing based on misincorporation and/or incorporation rates, as described elsewhere herein. Sequencing data was generated using a flow sequencing method that includes extending a primer hybridized to a template polynucleotide molecule according to a pre-determined flow cycle or flow order where, in any given flow position, a type of nucleotide base is accessible to the extending primer. A single type of nucleotide base was used in any given sequencing flow. A portion of the nucleotides of the particular base type included a fluorescent label, which, upon incorporation of the labeled nucleotides into the extending primer, renders a detectable signal.

Table 6 provides results of initial sequencing results for a subset of the polymerase mutants screened in accordance the flow sequencing method described above. Polymerase mutants for sequencing were selected in accordance with Example 3.

TABLE 6
Sequencing metrics for mutant polymerases.

| Mutation position | Polymerase # | Lag | Lead |
|---|---|---|---|
| D718K | 18 | 0.59 | 0.18 |
| n/a | 57 | 0.63 | 0.21 |
| V661I + L662M + | 75 | 1.18 | 0.87 |
| I68IV + T683K + | | | |
| M701L + F743Y + | | | |
| A800G + I689V + | | | |
| Q704I + V713M | | | |
| Q827D | 103 | 1.27 | 0.18 |
| K635S | 104 | 0.53 | 0.34 |
| R629A | 105 | 0.63 | 0.23 |
| R637M + L630P | 106 | 0.65 | 0 |
| I628L | 116 | 0.84 | 0.41 |
| I628M | 119 | 0.48 | 0.16 |
| V661I + L662M + | 127 | 1.56 | 1.27 |
| I681V + T683K + | | | |
| M701L + F710Y + | | | |
| A800G + 1689V | | | |
| N573E | 128 | 0.65 | 0.1 |
| L583N | 130 | 0.4 | 0.03 |
| L538F | 134 | 1.01 | 0.09 |
| Q579N | 135 | 0.4 | 0.05 |
| H568P | 142 | 0.42 | 0.15 |
| I628L + L583N | 149 | 1.02 | 0.24 |
| L630D | 151 | 0.66 | 0.11 |
| L630P | 152 | 0.84 | 0.22 |
| I628L + L630P | 158 | 0.16 | 0.05 |
| I628L + Q579N | 159 | 0.5 | 0.04 |
| A707Y | 160 | 0.44 | 0.03 |
| E572D | 162 | 3.64 | 0 |
| E623Y | 164 | 1.63 | 0.06 |
| I628L + L583V | 168 | 0.4 | 0.03 |
| I628L + L630D | 178 | 0.37 | 0 |
| R703A | 187 | 0.34 | 0 |
| I628M + D718A | 190 | 0.25 | 0.15 |
| I628M + D718K | 194 | 0.21 | 0 |
| I628M + R703A | 205 | 0.29 | 0.03 |
| R703A + D718K | 206 | 0.66 | 0 |
| I628M + R703A + | 207 | 0.7 | 0 |
| D718K | | | |
| V661I + L662M + | 228 | 0.51 | 0.05 |
| I68IV + T683K + | | | |
| M701L + F743Y + | | | |
| A800G + 1689V + | | | |
| Q704I + V713M + | | | |
| L630P | | | |
| I689F | 243 | 1.18 | 1.32 |
| V661I + L662M + | 247 | 1.51 | 1.04 |
| I681V + T683K + | | | |
| M701L + F710Y + | | | |
| A800G + I689V + | | | |
| L630P | | | |
| I689F + L630P | 248 | 1.29 | 0.97 |
| V661I + L662M + | 250 | 1.09 | 0.6 |
| I68IV + T683K + | | | |
| M701L + F743Y + | | | |
| A800G + I689V + | | | |
| Q704I + V713M + | | | |
| L630P + D718K | | | |
| I689F + L630P + | 252 | 1.65 | 0.72 |
| D718K | | | |

Lag is an indication of slower sequencing (e.g., due in some cases to a polymerase with a slower incorporation rate or a polymerase that dissociates at a higher rate from a template/primer complex). Lead is an indication of misincorporation (e.g., due to a polymerase that has a higher tolerance for incorrect incorporation errors). Pol57 and Pol75 served as a controls for the sequencing results in Table 6. Mutant polymerases with lower values for both lag and lead have improved sequencing activity. Thus, the polymerases with the I628L, L630P, and/or L630D mutation(s) have better overall lag/lead values in this set of sequencing results.

### Example 7: Additional Metrics for Evaluating Sequencing Results from Polymerase Mutants

Sequencing is a complex procedure, with many factors impacting the success of a sequencing run and many different features of the sequencing data being important for further analysis. Thus, there is no single one sequencing metric that completely determines the sequencing quality of a polymerase. In Example 6, lead and lag values are used as a proxy for sequencing quality, and these are very important indicators of polymerase efficacy. However, other sequencing metrics (e.g., metrics that are further downstream in the sequencing data analysis pipeline) can also be of use in evaluating mutant polymerases. In some cases, these additional metrics can be used in selecting a mutant polymerase for sequencing under a specific combination of conditions.

In some instances, a first mutant polymerase may exhibit improved sequencing quality under a first set of sequencing conditions and a second mutant polymerase may exhibit improved sequencing quality under a second set of conditions. Sequencing conditions may comprise types of nucleotide bases (e.g., labeled or not, type of label, linker used to attach label to nucleotide base, etc.), temperature, type of nucleic acid being sequenced (e.g., length, structural features, GC content, etc.), buffer conditions, or a combination thereof.

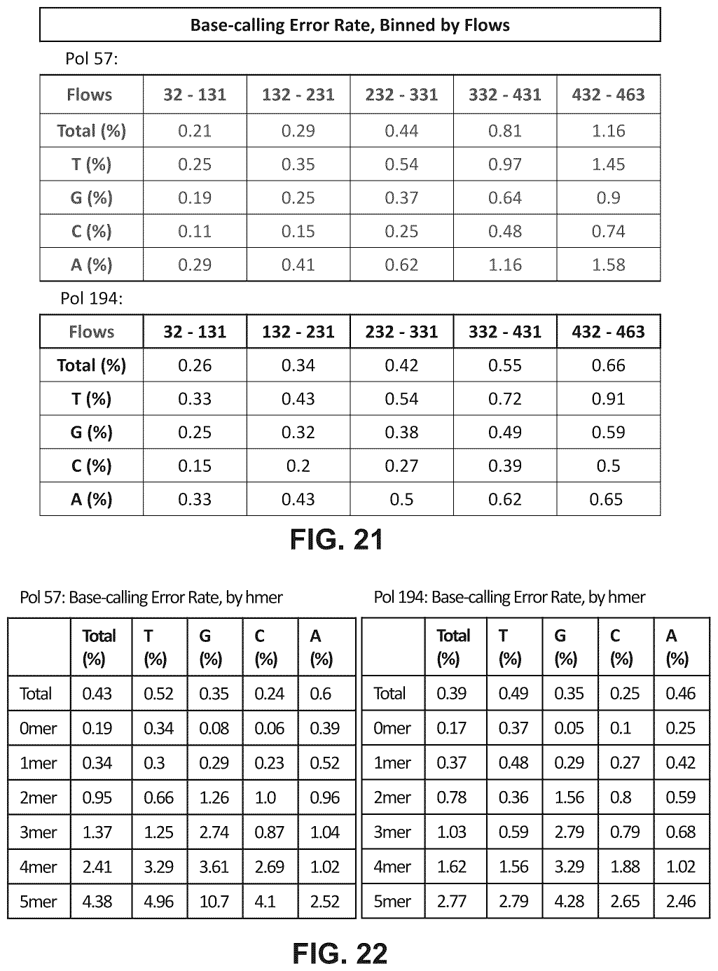

FIGS. 21 and 22 illustrate additional sequencing metrics from exemplary sequencing runs used for evaluating mutant polymerase 194, which has I628M+D718K mutations (Pol194). As shown in Table 6, Pol194 exhibited lag and lead values of 0.21 and 0, respectively. These indicated improved performance over the control Pol57. In these figures, the base error rates (i.e., the error in calling the correct nucleotide base at a loci) of Pol57 are compared with those of Pol194.

In FIG. 21, the base error rates are binned by nucleotide flow (i.e., for a flow-based sequencing approach). As the flows increase, the expectation is be for the error rate to rise, as there is an accumulation of polymerase errors (e.g., misincorporation) over time. As can be seen, Pol194 exhibited similar base error rates as Pol57 in at least the first half of the flows, and Pol194 had improved error rates (e.g., lower) over Pol57 in later flows (see e.g., flows 432-463). This data suggests that Pol194 does have improved sequencing performance and that this performance can be sustained over an entire sequencing run.

In FIG. 22, the base error rates are binned by homopolymer length (hmer). A homopolymer of length 4 (e.g., a 4mer) represents a sequence region with series of 4 of a same nucleotide base. Plate-based assays of multiple incorporation of a single base type (e.g., multiple C or multiple G incorporation shown in Example 5), can provide indications of whether a mutant polymerase has improved processivity over a control polymerase. Hmer base error rates indicate the quality of sequencing data, as potentially impacted by the processivity of the mutant polymerase.

Here, the top row of each table is a total of the base error rate for each nucleotide base type across all homopolymers. Pol194 overall has an improved base error rate over Pol57. In specific hmers, for each nucleotide base type, this improvement is sustained. The totality of these additional sequencing metrics helps in evaluating the efficacy of specific mutations.

### Example 8 FRET-Based Polymerase Assay with Non-Homopolymer Template Oligo

The following experiments were performed using a non-homopolymer template oligonucleotide (e.g., a template oligo that included a combination of nucleotide bases), and serve to confirm that: i) polymerases can handle incorporation of a variety of different labeled and non-labeled nucleotides, and ii) polymerases can perform nucleotide incorporation under high labeling conditions (i.e., where a larger percentage of the incorporated nucleotides are labeled).

In a first experiment, a primer (5′-GTTCCTGTCCACCTCC-3′, SEQ ID NO: 33) was annealed to a template oligo having the sequence 3′-CTCTCTCTCTCTCTCTCTCTG-5′ (i.e., a (CT)₁₀G oligo sequence, SEQ ID NO: 34) downstream from the 3′ end of the annealed primer (full length template sequence=5′-TCAGTCTCTCTCTCTCTCTCTCTCGGAGGTGGACAGGAAC-3′, SEQ ID NO: 35). None of the molecules (e.g., neither the primer nor the template oligo) are labeled. The primer and template oligonucleotide were dissolved to final concentrations of 15 nM and 10 nM, respectively, in a buffer of 20 mM Tris-HCl, pH 8.8, 2 mM MgCl2, 60 mM NaCl. A mutant polymerase (e.g., Pol 37; 40 nM final concentration) and an Atto 633-labeled dCTP were added to aliquots of the primer—template mixture, along with one of the following three nucleotide solutions (dNTPs at 500 nM final concentration):
A. dGTP/dATP-Atto532 (G/A* mix)
B. dGTP-Atto532/dATP (G*/A mix)
C. dGTP-Atto532/dATP-Atto532 (G*/A* mix),
   where * indicates a labeled dNTP.

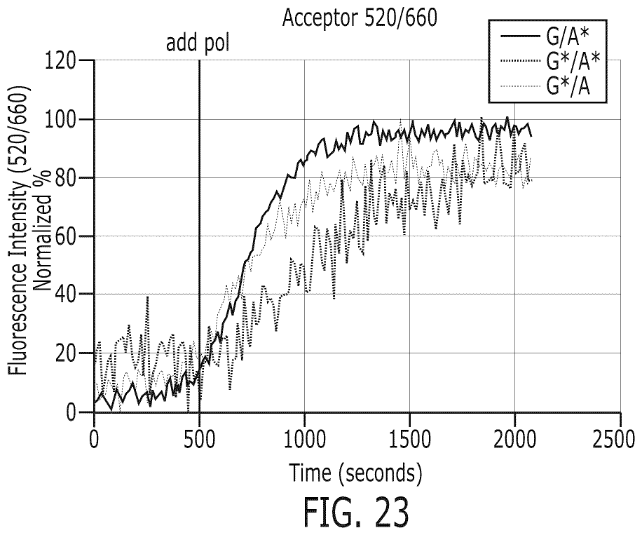

Aliquots of the reaction mixture were pipetted into individual wells of a 96-well microtiter plate, and the plate was placed in a fluorescence microplate reader pre-equilibrated to a temperature of 45° C. The mixtures were incubated in the microtiter plate at 45° C., and the fluorescence of the mixtures was monitored using 520 nm excitation light and a 660 nm emission wavelength setting. The normalized fluorescence responses following addition of the mutant polymerase are shown in FIG. 23. These fluorescence intensity traces demonstrate the sequential incorporation of multiple labeled nucleotides by a polymerase as detected by FRET using mixtures of Atto532 (donor)- and Atto633 (acceptor)-labeled dNTPs.

As can be seen in FIG. 23, the 520/660 nm fluorescence intensity increases over time for all three nucleotide mixtures (the magnitude of each fluorescence signal trace was normalized by setting the starting signal to 0%, and setting the highest signal ( ) to 100%). The rates of increase are approximately identical for the G/A* and G*/A mixtures, and is faster than for the G*/A* mixture. These observed fluorescence signal increases are indicative of successful enzymatic primer extension, where initially only Atto532-labeled nucleotides are incorporated into the complement of the poly-CT template sequence, followed eventually by the incorporation of the Atto633-labeled dCTP nucleotide opposite the G in the template. Note that a successful incorporation of the dCTP-Atto633 after one or more incorporated Atto532 labeled nucleotides is absolutely required for a 520/660 nm FRET signal to be measured. Only every other incorporated nucleotide is labeled with an Atto532 donor dye in the cases of the G/A* and G*/A mixtures, but with the G*/A* mix, the results show that a successful sequential incorporation of a total of 21 dye labeled nucleotides is possible under conditions of 100% labeling.

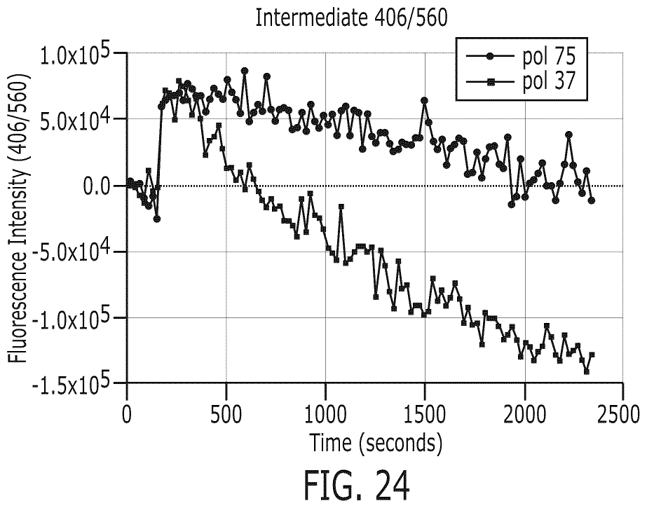

In a second experiment, the same primer/template hybrid was used as described above, but here the 5′-terminal T residue of the template strand was labeled with a Pacific Blue dye. Only a mixture of Atto532-labeled nucleotides was used (i.e., G*/A*), and fluorescence detection was performed using 406 nm excitation (to excite Pacific Blue molecules) and 560 nm emission wavelengths. With these fluorescence detection settings, incorporation of even a single Atto532-labeled nucleotide results in an increase in the 560 nm (Atto 532 emission) signal due to resonance energy transfer from excited Pacific Blue labels (the FRET donor). This can clearly be observed in FIG. 24, which illustrates the use of this three-dye system to evaluate the sequential incorporation of multiple dye labeled nucleotides by two different polymerases (note that only the fluorescence signal arising from incorporation of Atto532 labeled nucleotides—due to resonance energy transfer from excited Pacific Blue donor molecules—is shown in this figure; fluorescence signal intensities at t=zero were set to zero). After a successful fill-in reaction to complete the (CT)₁₀ portion of the template sequence, incorporation of Atto633-labeled dCTP results in a decrease of the 406/560 nm fluorescence as the newly-incorporated Atto633 dye (a FRET acceptor for Atto532 donors) quenches the emission of the Atto532 label. Again, this can clearly be observed in FIG. 24 following a period of relatively small or no change in the 406/560 nm emission, there is a steady decrease of the signal as the polymerases complete the primer extension reactions. Note that the two mutant polymerases used (Pol 37 and Pol 75) show significantly different reaction kinetics, with Pol 37 being faster than Pol 75.

It should be understood from the foregoing that, while particular implementations of the disclosed methods have been illustrated and described, various modifications can be made thereto and are contemplated herein. It is also not intended that the invention be limited by the specific examples provided within the specification. While the invention has been described with reference to the aforementioned specification, the descriptions and illustrations of the preferable embodiments herein are not meant to be construed in a limiting sense. Furthermore, it shall be understood that all aspects of the invention are not limited to the specific depictions, configurations or relative proportions set forth herein which depend upon a variety of conditions and variables. Various modifications in form and detail of the embodiments of the invention will be apparent to a person skilled in the art. It is therefore contemplated that the invention shall also cover any such modifications, variations, and equivalents.

What is claimed is:

1. A nucleic acid polymerase, comprising an amino acid sequence having at least 95% sequence identity to SEQ ID NO:1, wherein the polymerase comprises a mutation, relative to SEQ ID NO: 1, at an amino acid position selected from the group consisting of 495, 515, 526-534, 538, 549, 551-568, 570-590, 609-630, 635-637, 640, 655-663, 674, 681, 683, 688, 689, 701, 703-720, 728-730, 743, 761, 770-829, 872, and 873, wherein the amino acid position numbering is according to SEQ ID NO: 2.

2. The polymerase of claim 1, wherein the mutation is at amino acid position 495, 515, 529, 531, 538, 549, 558, 559, 561, 562, 563, 564, 565, 566, 567, 568, 572, 573, 575, 579, 580, 583, 622, 623, 628, 629, 630, 635, 636, 637, 640, 655, 660, 661, 662, 674, 681, 683, 688, 689, 701, 703, 704, 707, 710, 713, 716, 718, 743, 761, 780, 782, 785, 786, 788, 800, 806, 815, 819, 826, 827, 872, or 873.

3. The polymerase of claim 2, wherein the mutation is V495F, E515K, N529R, P531Q, L538F, K549I, A558M, D559K, D559R, D559S, L561I, E562N, K563G, L564V, A565R, P566L, Y567H, H568P, E572D, N573E, L575I, Q579N, L580D, L580G, L583N, L583V, N622G, E623Y, I628L, I628M, R629A, L630D, L630P, K635S, I636M, I636L, R637M, F640V, S655N, R660C, V661I, L662M, A674V, I681V, T683K, T683V, D688M, I689F, I689Q, I689V, I689Y, M701L, R703A, R703H, R703P, Q704I, A707Y, F710Y, V713M, I716Q, D718A, D718K, F743R, F743Y, G761E, N780K, N782R, N782V, S785R, F786R, E788R, A800G, K806E, K806M, L815F, R819K, R819M, R819P, R819V, L826Y, Q827D, W872H, or Y873D.

4. The polymerase of claim 1, wherein the polymerase comprises at least two mutations, relative to SEQ ID NO: 1, at amino acid positions selected from the group consisting of 495, 515, 526-534, 538, 549, 551-568, 570-590, 609-630, 635-637, 640, 655-663, 674, 681, 683, 688, 689, 701, 703-720, 728-730, 743, 761, 770-829, 872, and 873.

5. The polymerase of claim 4, wherein the at least two mutations are at amino acid positions 495, 515, 529, 531, 538, 549, 558, 559, 561, 562, 563, 564, 565, 566, 567, 568, 572, 573, 575, 579, 580, 583, 622, 623, 628, 629, 630, 635, 636, 637, 640, 655, 660, 661, 662, 674, 681, 683, 688, 689, 701, 703, 704, 707, 710, 713, 716, 718, 743, 761, 780, 782, 785, 786, 788, 800, 806, 815, 819, 826, 827, 872, or 873.

6. The polymerase of claim 5, wherein the at least two mutations comprise two or more of V495F, E515K, N529R, P531Q, L538F, K549I, A558M, D559K, D559R, D559S, L561I, E562N, K563G, L564V, A565R, P566L, Y567H, H568P, E572D, N573E, L575I, Q579N, L580D, L580G, L583N, L583V, N622G, E623Y, I628L, I628M, R629A, L630D, L630P, K635S, I636M, I636L, R637M, F640V, S655N, R660C, V661I, L662M, A674V, 1681V, T683K, T683V, D688M, I689F, I689Q, I689V, I689Y, M701L, R703A, R703H, R703P, Q704I, A707Y, F710Y, V713M, I716Q, D718A, D718K, F743R, F743Y, G761E, N780K, N782R, N782V, S785R, F786R, E788R, A800G, K806E, K806M, L815F, R819K, R819M, R819P, R819V, L826Y, Q827D, W872H, or Y873D.

7. The polymerase of any one of claims 1-6, wherein the amino acid sequence comprises at least 95% identity to SEQ ID NO: 2.

8. The polymerase of any one of claims 1-6, wherein the amino acid sequence further comprises a DNA binding domain.

9. The polymerase of claim 8, wherein the DNA binding domain comprises a sequence at least 95% identical to SEQ ID NO: 4 or SEQ ID NO: 5.

10. The polymerase of claim 8, wherein the DNA binding domain comprises a sequence of SEQ ID NO: 4 or SEQ ID NO: 5.

11. The polymerase of any one of claims 8-10, wherein the DNA binding domain is attached to the N-terminus of a polymerase domain.

12. The polymerase of claim 11, wherein the DNA binding domain is attached to the N-terminus of the polymerase domain through an amino acid linker.

13. A nucleic acid polymerase, comprising an amino acid sequence having at least 95% sequence identity to SEQ ID NO: 3, wherein the polymerase comprises a mutation, relative to SEQ ID NO: 3, at an amino acid position selected from the group consisting of 495, 515, 526-534, 538, 549, 551-568, 570-590, 609-630, 635-637, 640, 655-663, 674, 681, 683, 688, 689, 701, 703-720, 728-730, 743, 761, 770-829, 872, and 873, wherein the amino acid position numbering is according to SEQ ID NO: 2.

14. The polymerase of claim 13, wherein the mutation is at amino acid position 495, 515, 529, 531, 538, 549, 558, 559, 561, 562, 563, 564, 565, 566, 567, 568, 572, 573, 575, 579, 580, 583, 622, 623, 628, 629, 630, 635, 636, 637, 640, 655, 660, 661, 662, 674, 681, 683, 688, 689, 701, 703, 704, 707, 710, 713, 716, 718, 743, 761, 780, 782, 785, 786, 788, 800, 806, 815, 819, 826, 827, 872, or 873, wherein the amino acid position numbering is according to SEQ ID NO: 2.

15. The polymerase of claim 13, wherein the mutation is V495F, E515K, N529R, P531Q, L538F, K549I, A558M, D559K, D559R, D559S, L561I, E562N, K563G, L564V, A565R, P566L, Y567H, H568P, E572D, N573E, L575I, Q579N, L580D, L580G, L583N, L583V, N622G, E623Y, I628L, I628M, R629A, L630D, L630P, K635S, I636M, I636L, R637M, F640V, S655N, R660C, V661I, L662M, A674V, I681V, T683K, T683V, D688M, I689F, I689Q, I689V, I689Y, M701L, R703A, R703H, R703P, Q704I, A707Y, F710Y, V713M, I716Q, D718A, D718K, F743R, F743Y, G761E, N780K, N782R, N782V, S785R, F786R, E788R, A800G, K806E, K806M, L815F, R819K, R819M, R819P, R819V, L826Y, Q827D, W872H, or Y873D, wherein the amino acid position numbering is according to SEQ ID NO: 2.

16. The polymerase of claim 13, wherein the polymerase comprises at least two mutations, relative to SEQ ID NO: 1, at amino acid positions selected from the group consisting of 495, 515, 526-534, 538, 549, 551-568, 570-590, 609-630, 635-637, 640, 655-663, 674, 681, 683, 688, 689, 701, 703-720, 728-730, 743, 761, 770-829, 872, and 873, wherein the amino acid position numbering is according to SEQ ID NO: 2.

17. The polymerase of claim 16, wherein the at least two mutations are at amino acid positions 495, 515, 529, 531, 538, 549, 558, 559, 561, 562, 563, 564, 565, 566, 567, 568, 572, 573, 575, 579, 580, 583, 622, 623, 628, 629, 630, 635, 636, 637, 640, 655, 660, 661, 662, 674, 681, 683, 688, 689, 701, 703, 704, 707, 710, 713, 716, 718, 743, 761, 780, 782, 785, 786, 788, 800, 806, 815, 819, 826, 827, 872, or 873, wherein the amino acid position numbering is according to SEQ ID NO: 2.

18. The polymerase of claim 16, wherein the at least two mutations comprise two or more of V495F, E515K, N529R, P531Q, L538F, K549I, A558M, D559K, D559R, D559S, L561I, E562N, K563G, L564V, A565R, P566L, Y567H, H568P, E572D, N573E, L575I, Q579N, L580D, L580G, L583N, L583V, N622G, E623Y, I628L, I628M, R629A, L630D, L630P, K635S, I636M, I636L, R637M, F640V, S655N, R660C, V661I, L662M, A674V, I681V, T683K, T683V, D688M, I689F, I689Q, I689V, I689Y, M701L, R703A, R703H, R703P, Q704I, A707Y, F710Y, V713M, I716Q, D718A, D718K, F743R, F743Y, G761E, N780K, N782R, N782V, S785R, F786R, E788R, A800G, K806E, K806M, L815F, R819K, R819M, R819P, R819V, L826Y, Q827D, W872H, or Y873D, wherein the amino acid position numbering is according to SEQ ID NO: 2.

19. The polymerase of any one of claims 1-18, wherein the polymerase comprises a mutation at amino acid position 628, wherein the amino acid position numbering is according to SEQ ID NO: 2.

20. The polymerase of claim 19, wherein the polymerase comprises a I628L mutation, wherein the amino acid position numbering is according to SEQ ID NO: 2.

21. The polymerase of claim 20, wherein the polymerase comprises a 785R mutation, wherein the amino acid position numbering is according to SEQ ID NO: 2.

22. The polymerase of any one of claims 1-18, wherein the polymerase comprises a mutation at amino acid position 630, wherein the amino acid position numbering is according to SEQ ID NO: 2.

23. The polymerase of claim 22, wherein the polymerase comprises a L630D mutation, wherein the amino acid position numbering is according to SEQ ID NO: 2.

24. The polymerase of claim 22, wherein the polymerase comprises a L630P mutation, wherein the amino acid position numbering is according to SEQ ID NO: 2.

25. The polymerase of any one of claims 1-18, wherein the polymerase comprises a mutation at amino acid position 785, wherein the amino acid position numbering is according to SEQ ID NO: 2.

26. The polymerase of claim 25, wherein the polymerase comprises an S785R mutation, wherein the amino acid position numbering is according to SEQ ID NO: 2.

27. The polymerase of any one of claims 1-18, wherein the polymerase comprises a mutation at amino acid position 827, wherein the amino acid position numbering is according to SEQ ID NO: 2.

28. The polymerase of claim 27, wherein the polymerase comprises a Q827D mutation, wherein the amino acid position numbering is according to SEQ ID NO: 2.

29. A composition comprising the polymerase of any one of claims 1-28, and an aqueous solution.

30. The composition of claim 29, further comprising nucleotides and a nucleic acid hybrid comprising a target nucleic acid molecule hybridized to a sequencing primer.

31. The composition of claim 30, wherein at least a portion of the nucleotides are labeled nucleotides.

32. The composition of claim 31, wherein the labeled nucleotides comprise a fluorescent label.

33. A nucleic acid molecule encoding the polymerase of any one of claims 1-28.

34. An expression vector comprising the nucleic acid molecule of claim 33.

35. A host cell, comprising the expression vector of claim 34.

36. A method of making a polymerase, comprising:
culturing the host cell of claim 35;
expressing, using the host cell, the polymerase; and
isolating the polymerase.

37. A method, comprising: providing in a reaction mixture (i) a nucleic acid molecule, (ii) a labeled nucleotide, and (iii) the mutant polymerase of any one of claims 1-28, under conditions sufficient to extend the nucleic acid molecule with the labeled nucleotide.

38. A method, comprising: contacting a labeled nucleotide with the mutant polymerase of any one of claims 1-28 and extending a nucleic acid molecule to incorporate the labeled nucleotide.

39. The method of claims 37 or 38, wherein said labeled nucleotide comprises a fluorescent dye.


SEQUENCE LISTING (35 sequences)

SEQ ID NO: 1 - protein, 580 aa, synthetic construct
AKMAFTLADRVTEEMLADKAALVVEVVEENYHDAPIVGIAVVNEHGRFFLRPETALADPQFVAWLGDETKKKSMFDSKRAAVALKWKGIELCGVSFDLLLAAYLLDPAQGVDDVAAAAKMKQYEAVRPDEAVYGKGAKRAVPDEPVLAEHLVRKAAAIWELERPFLDELRRNEQDRLLVELEQPLSSILAEMEFAGVKVDTKRLEQMGKELAEQLGTVEQRIYELAGQEFNINSPKQLGVILFEKLQLPVLKKTKTGYSTSADVLEKLAPYHEIVENILHYRQLGKLQSTYIEGLLKVVRPDTKKVHTIFNQALTQTGRLSSTEPNLQNIPIRLEEGRKIRQAFVPSESDWLIFAADYSQIELRVLAHIAEDDNLMEAFRRDLDIHTKTAMDIFQVSEDEVTPNMRRQAKAVNFGIVYGISDYGLAQNLNISRKEAAEFIERYFESFPGVKRYMENIVQEAKQKGYVTTLLHRRRYLPDITSRNFNVRSFAERMAMNTPIQGSAADIIKKAMIDLNARLKEERLQAHLLLQVHDELILEAPKEEMERLCRLVPEVMEQAVTLRVPLKVDYHYGSTWYDAK

SEQ ID NO: 2 - protein, 876 aa, synthetic construct
MKKKLVLIDGSSVAYRAFFALPLLHNDKGIHTNAVYGFTMMLNKILAEEEPTHMLVAFDAGKTTFRHEAFQEYKGGRQQTPPELSEQFPLLRELLRAYRIPAYELENYEADDIIGTLAARAEQEGFEVKVISGDRDLTQLASPHVTVDITKKGITDIEPYTPETVREKYGLTPEQIVDLKGLMGDKSDNIPGVPGIGEKTAVKLLKQFGTVENVLASIDEIKGEKLKETLRQHREMALLSKKLAAIRRDAPVELSLDDIVYQGEDREKVVALFKELGFQSFLEKMESPSSEEEKPLAKMAFTLADRVTEEMLADKAALVVEVVEENYHDAPIVGIAVVNEHGRFFLRPETALADPQFVAWLGDETKKKSMFDSKRAAVALKWKGIELCGVSFDLLLAAYLLDPAQGVDDVAAAAKMKQYEAVRPDEAVYGKGAKRAVPDEPVLAEHLVRKAAAIWELERPFLDELRRNEQDRLLVELEQPLSSILAEMEFAGVKVDTKRLEQMGKELAEQLGTVEQRIYELAGQEFNINSPKQLGVILFEKLQLPVLKKTKTGYSTSADVLEKLAPYHEIVENILHYRQLGKLQSTYIEGLLKVVRPDTKKVHTIFNQALTQTGRLSSTEPNLQNIPIRLEEGRKIRQAFVPSESDWLIFAADYSQIELRVLAHIAEDDNLMEAFRRDLDIHTKTAMDIFQVSEDEVTPNMRRQAKAVNFGIVYGISDYGLAQNLNISRKEAAEFIERYFESFPGVKRYMENIVQEAKQKGYVTTLLHRRRYLPDITSRNFNVRSFAERMAMNTPIQGSAADIIKKAMIDLNARLKEERLQARLLLQVHDELILEAPKEEMERLCRLVPEVMEQAVTLRVPLKVDYHYGSTWYDAK

SEQ ID NO: 3 - protein, 666 aa, synthetic construct
MATKIKFKYKGQDLEVDISKVKKVWKVGKMVSFTYDDNGKTGRGAVSEKDAPKELLSMIGKKGGGGSGGGGGSESPSSEEEKPLAKMAFTLADRVTEEMLADKAALVVEVVEENYHDAPIVGIAVVNEHGRFFLRPETALADPQFVAWLGDETKKKSMFDSKRAAVALKWKGIELCGVSFDLLLAAYLLDPAQGVDDVAAAAKMKQYEAVRPDEAVYGKGAKRAVPDEPVLAEHLVRKAAAIWELERPFLDELRRNEQDRLLVELEQPLSSILAEMEFAGVKVDTKRLEQMGKELAEQLGTVEQRIYELAGQEFNINSPKQLGVILFEKLQLPVLKKTKTGYSTSADVLEKLAPYHEIVENILHYRQLGKLQSTYIEGLLKVVRPDTKKVHTIFNQALTQTGRLSSTEPNLQNIPIRLEEGRKIRQAFVPSESDWLIFAADYSQIELRVLAHIAEDDNLMEAFRRDLDIHTKTAMDIFQVSEDEVTPNMRRQAKAVNFGIVYGISDYGLAQNLNISRKEAAEFIERYFESFPGVKRYMENIVQEAKQKGYVTTLLHRRRYLPDITSRNFNVRSFAERMAMNTPIQGSAADIIKKAMIDLNARLKEERLQARLLLQVHDELILEAPKEEMERLCRLVPEVMEQAVTLRVPLKVDYHYGSTWYDAKLE

SEQ ID NO: 4 - protein, 62 aa, synthetic construct
MATKIKFKYKGQDLEVDISKVKKVWKVGKMVSFTYDDNGKTGRGAVSEKDAPKELLSMIGKK

SEQ ID NO: 5 - protein, 63 aa, synthetic construct
MATVKFKYKGEEKQVDISKIKKVWRVGKMISFTYDEGGGKTGRGAVSEKDAPKELLQMLEKQK

SEQ ID NO: 6 - protein, 673 aa, synthetic construct
MATVKFKYKGEEKQVDISKIKKVWRVGKMISFTYDEGGGKTGRGAVSEKDAPKELLQMLEKQKGGGGSGGGGGSGGGGGSESPSSEEEKPLAKMAFTLADRVTEEMLADKAALVVEVVEENYHDAPIVGIAVVNEHGRFFLRPETALADPQFVAWLGDETKKKSMFDSKRAAVALKWKGIELCGVSFDLLLAAYLLDPAQGVDDVAAAAKMKQYEAVRPDEAVYGKGAKRAVPDEPVLAEHLVRKAAAIWELERPFLDELRRNEQDRLLVELEQPLSSILAEMEFAGVKVDTKRLEQMGKELAEQLGTVEQRIYELAGQEFNINSPKQLGVILFEKLQLPVLKKTKTGYSTSADVLEKLAPYHEIVENILHYRQLGKLQSTYIEGLLKVVRPDTKKVHTIFNQALTQTGRLSSTEPNLQNIPLRLEEGRKIRQAFVPSESDWLIFAADYSQIELRVLAHIAEDDNLMEAFRRDLDIHTKTAMDIFQVSEDEVTPNMRRQAKAVNFGIVYGISDYGLAQNLNISRKEAAEFIERYFESFPGVKRYMENIVQEAKQKGYVTTLLHRRRYLPDITSRNFNVRRFAERMAMNTPIQGSAADIIKKAMIDLNARLKEERLQARLLLQVHDELILEAPKEEMERLCRLVPEVMEQAVTLRVPLKVDYHYGSTWYDAKLE

SEQ ID NO: 7 - protein, 666 aa, synthetic construct
MATKIKFKYKGQDLEVDISKVKKVWKVGKMVSFTYDDNGKTGRGAVSEKDAPKELLSMIGKKGGGGSGGGGGSESPSSEEEKPLAKMAFTLADRVTEEMLADKAALVVEVVEENYHDAPIVGIAVVNEHGRFFLRPETALADPQFVAWLGDETKKKSMFDSKRAAVALKWKGIELCGVSFDLLLAAYLLDPAQGVDDVAAAAKMKQYEAVRPDEAVYGKGAKRAVPDEPVLAEHLVRKAAAIWELERPFLDELRRNEQDRLLVELEQPLSSILAEMEFAGVKVDTKRLEQMGKELAEQLGTVEQRIYELAGQEFNINSPKQLGVILFEKLQLPVLKKTKTGYSTSADVLEKLAPYHEIVENILHYRQLGKLQSTYIEGLLKVVRPDTKKVHTIFNQALTQTGRLSSTEPNLQNIPLRLEEGRKIRQAFVPSESDWLIFAADYSQIELRVLAHIAEDDNLMEAFRRDLDIHTKTAMDIFQVSEDEVTPNMRRQAKAVNFGIVYGISDYGLAQNLNISRKEAAEFIERYFESFPGVKRYMENIVQEAKQKGYVTTLLHRRRYLPDITSRNFNVRSFAERMAMNTPIQGSAADIIKKAMIDLNARLKEERLQARLLLQVHDELILEAPKEEMERLCRLVPEVMEQAVTLRVPLKVDYHYGSTWYDAKLE

SEQ ID NO: 8 - protein, 666 aa, synthetic construct
MATKIKFKYKGQDLEVDISKVKKVWKVGKMVSFTYDDNGKTGRGAVSEKDAPKELLSMIGKKGGGGSGGGGGSESPSSEEEKPLAKMAFTLADRVTEEMLADKAALVVEVVEENYHDAPIVGIAVVNEHGRFFLRPETALADPQFVAWLGDETKKKSMFDSKRAAVALKWKGIELCGVSFDLLLAAYLLDPAQGVDDVAAAAKMKQYEAVRPDEAVYGKGAKRAVPDEPVLAEHLVRKAAAIWELERPFLDELRRNEQDRLLVELEQPLSSILAEMEFAGVKVDTKRLEQMGKELAEQLGTVEQRIYELAGQEFNINSPKQLGVILFEKLQLPVLKKTKTGYSTSADVLEKLAPYHEIVENILHYRQLGKLQSTYIEGLLKVVRPDTKKVHTIFNQALTQTGRLSSTEPNLQNIPIRDEEGRKIRQAFVPSESDWLIFAADYSQIELRVLAHIAEDDNLMEAFRRDLDIHTKTAMDIFQVSEDEVTPNMRRQAKAVNFGIVYGISDYGLAQNLNISRKEAAEFIERYFESFPGVKRYMENIVQEAKQKGYVTTLLHRRRYLPDITSRNFNVRSFAERMAMNTPIQGSAADIIKKAMIDLNARLKEERLQARLLLQVHDELILEAPKEEMERLCRLVPEVMEQAVTLRVPLKVDYHYGSTWYDAKLE

SEQ ID NO: 9 - protein, 666 aa, synthetic construct
MATKIKFKYKGQDLEVDISKVKKVWKVGKMVSFTYDDNGKTGRGAVSEKDAPKELLSMIGKKGGGGSGGGGGSESPSSEEEKPLAKMAFTLADRVTEEMLADKAALVVEVVEENYHDAPIVGIAVVNEHGRFFLRPETALADPQFVAWLGDETKKKSMFDSKRAAVALKWKGIELCGVSFDLLLAAYLLDPAQGVDDVAAAAKMKQYEAVRPDEAVYGKGAKRAVPDEPVLAEHLVRKAAAIWELERPFLDELRRNEQDRLLVELEQPLSSILAEMEFAGVKVDTKRLEQMGKELAEQLGTVEQRIYELAGQEFNINSPKQLGVILFEKLQLPVLKKTKTGYSTSADVLEKLAPYHEIVENILHYRQLGKLQSTYIEGLLKVVRPDTKKVHTIFNQALTQTGRLSSTEPNLQNIPIRPEEGRKIRQAFVPSESDWLIFAADYSQIELRVLAHIAEDDNLMEAFRRDLDIHTKTAMDIFQVSEDEVTPNMRRQAKAVNFGIVYGISDYGLAQNLNISRKEAAEFIERYFESFPGVKRYMENIVQEAKQKGYVTTLLHRRRYLPDITSRNFNVRSFAERMAMNTPIQGSAADIIKKAMIDLNARLKEERLQARLLLQVHDELILEAPKEEMERLCRLVPEVMEQAVTLRVPLKVDYHYGSTWYDAKLE

SEQ ID NO: 10 - protein, 666 aa, synthetic construct
MATKIKFKYKGQDLEVDISKVKKVWKVGKMVSFTYDDNGKTGRGAVSEKDAPKELLSMIGKKGGGGSGGGGGSESPSSEEEKPLAKMAFTLADRVTEEMLADKAALVVEVVEENYHDAPIVGIAVVNEHGRFFLRPETALADPQFVAWLGDETKKKSMFDSKRAAVALKWKGIELCGVSFDLLLAAYLLDPAQGVDDVAAAAKMKQYEAVRPDEAVYGKGAKRAVPDEPVLAEHLVRKAAAIWELERPFLDELRRNEQDRLLVELEQPLSSILAEMEFAGVKVDTKRLEQMGKELAEQLGTVEQRIYELAGQEFNINSPKQLGVILFEKLQLPVLKKTKTGYSTSADVLEKLAPYHEIVENILHYRQLGKLQSTYIEGLLKVVRPDTKKVHTIFNQALTQTGRLSSTEPNLQNIPIRLEEGRKIRQAFVPSESDWLIFAADYSQIELRVLAHIAEDDNLMEAFRRDLDIHTKTAMDIFQVSEDEVTPNMRRQAKAVNFGIVYGISDYGLAQNLNISRKEAAEFIERYFESFPGVKRYMENIVQEAKQKGYVTTLLHRRRYLPDITSRNFNVRSFAERMAMNTPIQGSAADIIKKAMIDLNARLKEERLQARLLLDVHDELILEAPKEEMERLCRLVPEVMEQAVTLRVPLKVDYHYGSTWYDAKLE

SEQ ID NO: 11 - protein, 576 aa, synthetic construct
FTLADRVTEEMLADKAALVVEVVEENYHDAPIVGIAVVNEHGRFFLRPETALADPQFVAWLGDETKKKSMFDSKRAAVALKWKGIELCGVSFDLLLAAYLLDPAQGVDDVAAAAKMKQYEAVRPDEAVYGKGAKRAVPDEPVLAEHLVRKAAAIWELERPFLDELRRNEQDRLLVELEQPLSSILAEMEFAGVKVDTKRLEQMGKELAEQLGTVEQRIYELAGQEFNINSPKQLGVILFEKLQLPVLKKTKTGYSTSADVLEKLAPYHEIVENILHYRQLGKLQSTYIEGLLKVVRPDTKKVHTIFNQALTQTGRLSSTEPNLQNIPIRLEEGRKIRQAFVPSESDWLIFAADYSQIELRVLAHIAEDDNLMEAFRRDLDIHTKTAMDIFQVSEDEVTPNMRRQAKAVNFGIVYGISDYGLAQNLNISRKEAAEFIERYFESFPGVKRYMENIVQEAKQKGYVTTLLHRRRYLPDITSRNFNVRSFAERMAMNTPIQGSAADIIKKAMIDLNARLKEERLQARLLLQVHDELILEAPKEEMERLCRLVPEVMEQAVTLRVPLKVDYHYGSTWYDAK

SEQ ID NO: 12 - protein, 11 aa, synthetic construct
GGGGSGGGGGS

SEQ ID NO: 13 - protein, 11 aa, synthetic construct
ESPSSEEEKPL

SEQ ID NO: 14 - protein, 17 aa, synthetic construct
GGGGSGGGGGSGGGGGS

SEQ ID NO: 15 - protein, 66 aa, synthetic construct
MVKVKFKYKGEEKEVDTSKIKKVWRVGKMVSFTYDDNGKTGRGAVSEKDAPKELLDMLARAEREKK

SEQ ID NO: 16 - protein, 65 aa, synthetic construct
MAKVRFKYKGEEKEVDTSKIKKVWRVGKMVSFTYDDNGKTGRGAVSEKDAPKELMDMLARAEKKK

SEQ ID NO: 17 - protein, 59 aa, synthetic construct
MVKVKFKYKGEEKEVDTSKIKKVWRAGKAVSFTYDDNGKTGRGAVSEKDAPKELLDMLA

SEQ ID NO: 18 - protein, 59 aa, synthetic construct
MVKVKFKYKGEEKEVDTSKIKKVWRFGKFVSFTYDDNGKTGRGAVSEKDAPKELLDMLA

SEQ ID NO: 19 - protein, 59 aa, synthetic construct
MVSIKFKYKGEEKEVDISKIKKVWKVGNMVSFTYDDNGKTGRGALSLKEAPQELLQKLN

SEQ ID NO: 20 - protein, 61 aa, synthetic construct
ATVKFTHQGEEKQVDISKIKWVIRWGQYIWFKYDEDGGAKGWGYVSEKDAPKELLQMLKKR

SEQ ID NO: 21 - protein, 61 aa, synthetic construct
VKFVSSGEEKEVDTSKIKKVWRNLTKYGTIVQFTYDDNGKTGRGYVRELDAPKELLDMLAR

SEQ ID NO: 22 - protein, 62 aa, synthetic construct
MATKIKFKYKGQDLEVDISKVKKVWKVGKMVSFTYDDNGKTGRGAVSEKDAPKELLNMIGKK

SEQ ID NO: 23 - protein, 60 aa, synthetic construct
VKFFWNGEEKEVDTSKIVWVKRAGKSVLFIYDDNGKNGYGDVTEKDAPKELLDMLARAER

SEQ ID NO: 24 - protein, 60 aa, synthetic construct
VKFLLNGEEKEVDTSKIRDVSRQGKNVKFLYNDNGKYGAGNVDEKDAPKELLDXLARAER

SEQ ID NO: 25 - DNA, 15 bp, synthetic construct
  source 1..15: mol_type = other DNA
tttatcgaccagact

SEQ ID NO: 26 - DNA, 13 bp, synthetic construct
  source 1..13: mol_type = other DNA
tttcgtcagcact

SEQ ID NO: 27 - DNA, 14 bp, synthetic construct
  source 1..14: mol_type = other DNA
ttttttgtccgact

SEQ ID NO: 28 - DNA, 15 bp, synthetic construct
  source 1..15: mol_type = other DNA
tctaaattagggact

SEQ ID NO: 29 - DNA, 10 bp, synthetic construct
  source 1..10: mol_type = other DNA
accccagcct

SEQ ID NO: 30 - DNA, 11 bp, synthetic construct
  source 1..11: mol_type = other DNA
acccccagcct

SEQ ID NO: 31 - DNA, 11 bp, synthetic construct
  source 1..11: mol_type = other DNA
tttcagggcag

SEQ ID NO: 32 - DNA, 11 bp, synthetic construct
  source 1..11: mol_type = other DNA
tttgaccccag

SEQ ID NO: 33 - DNA, 16 bp, synthetic construct
  source 1..16: mol_type = other DNA
gttcctgtccacctcc

SEQ ID NO: 34 - DNA, 21 bp, synthetic construct
  source 1..21: mol_type = other DNA
gtctctctctctctctctctc

SEQ ID NO: 35 - DNA, 40 bp, synthetic construct
  source 1..40: mol_type = other DNA
tcagtctctctctctctctctctcggaggtggacaggaac